SEQUENCE LISTING (10 sequences)

SEQ ID NO: 1 - DNA, 657 bp, Homo sapiens
  CDS (1)...(657)
atgtttgagaggcgcaaagagcaagacgagggaccaggaatccatgaatttcttgcatttttatgggctgaattgggctctgaagccaaagaagagaaagaagaagaacggacctgccggttgctgggcaagtgtgtagatgccgaaaaccattcccttgttattggaggactgtttcctattgactccaggaccatcccagcaaatgagtctattttggagccagcatcagcaaaatgtgaagggtttaactttcagagattccgctggatgaaagccatgatccacatgatcaaggagattaataagaggaaggatattttgcccaacatcactctgggctatcagatctttgatacctgttttaccatctccaaatcagtggaagcagtcttggtatttcttacagggcaggaagaaaacaggcccaattttagaaacagcactggagcatttccggcaggaattgttggagcaggtggatcattcttatcagttcctgcttcaagaattctagggttatattatttgcctcaggtgggctatacctctacctgcgtgattcttagtgacaaataccagtttccatcttatcttcgtgtaatagccagcgataagatccagtcgaaggctgtggtaaaacgtatccaacacttt

SEQ ID NO: 2 - protein, 219 aa, Homo sapiens
MFERRKEQDEGPGIHEFLAFLWAELGSEAKEEKEEERTCRLLGKCVDAENHSLVIGGLFPIDSRTIPANESILEPASAKCEGFNFQRFRWMKAMIHMIKEINKRKDILPNITLGYQIFDTCFTISKSVEAVLVFLTGQEENRPNFRNSTGAFPAGIVGAGGSFLSVPASRILGLYYLPQVGYTSTCVILSDKYQFPSYLRVIASDKIQSKAVVKRIQHF

SEQ ID NO: 3 - DNA, 657 bp, Artificial Sequence
  This degenerate nucleotide sequence encodes the amino acid sequence of SEQ ID NO2.
atgttygarmgnmgnaargarcargaygarggnccnggnathcaygarttyytngcnttyytntgggcngarytnggnwsngargcnaargargaraargargargarmgnacntgymgnytnytnggnaartgygtngaygcngaraaycaywsnytngtnathggnggnytnttyccnathgaywsnmgnacnathccngcnaaygarwsnathytngarccngcnwsngcnaartgygarggnttyaayttycarmgnttymgntggatgaargcnatgathcayatgathaargarathaayaarmgnaargayathytnccnaayathacnytnggntaycarathttygayacntgyttyacnathwsnaarwsngtngargcngtnytngtnttyytnacnggncargargaraaymgnccnaayttymgnaaywsnacnggngcnttyccngcnggnathgtnggngcnggnggnwsnttyytnwsngtnccngcnwsnmgnathytnggnytntaytayytnccncargtnggntayacnwsnacntgygtnathytnwsngayaartaycarttyccnwsntayytnmgngtnathgcnwsngayaarathcarwsnaargcngtngtnaarmgnathcarcaytty

SEQ ID NO: 4 - DNA, 1140 bp, Homo sapiens
  CDS (1)...(1140)
cttccccattcagtgtgtactgatgtgtgtcctcctgggactggaaggggattcgttcagagggaaccaatatgctgctttgactccatcccatgtgctgatggacacgtgtcacggaaaccaggtgaaagggagtgtgaacaatgtggtgaagactattggtcaaatgcacaaaagagcgagtgtgtgctgaaagaggtggaataccttgcttatgatgaggccctgggattcacacttgtcattctttctgtctttggggcatttgtggtcttggcagtcacagctgtgtatgtgatacacaggcacactcccctggtgaacgccagtgactggcagctgggctttctcattcaggtttctctgatcatcatgctgctgtcgtccatgcttttcattgacaagccacacaactggtcctgcatggctggccaggtcactctggcactgggcttttctctttgcctgtcttgccttcttggaaagactagttcactgtttttagcctacagaatttccaaatccaaaactcaacttacatccatgcaccccctttatcggaaaatcattgtgctaatctctgttctagcggagattggcatatgtacagcctacttgatattggaacctcccatggtatacaagaacatggaatctcaaaatacaaagatcattctgggatgcaatgaaatttccatagagtttttgtactcgatgtttggaattgatgccttcttagccttgctatgctttcttacaacttttgtggctcgccagttaccagataattactatgaaggaaaatgcatcacctttgggatgcttgtctttttcatcatttggatgtcttttgtccctgtttatttgagcaccaaaggcaagttcaaaatggctgtggaaatatttgcaatcttggcatccagccatggcttgttgggttgtatatttgctcctaagtgcctcattattttgctgaggccagagaggaacaccagtgaaattgtttgtggaagagtctccaccacagataattgcatccaactgacctcagcttttgtgagcagtgagcttaacaataccacagtgtcaactgttctggatgacagagttttgatttacatgtgtcctttgaagctgcaa

SEQ ID NO: 5 - protein, 380 aa, Homo sapiens
LPHSVCTDVCPPGTGRGFVQREPICCFDSIPCADGHVSRKPGERECEQCGEDYWSNAQKSECVLKEVEYLAYDEALGFTLVILSVFGAFVVLAVTAVYVIHRHTPLVNASDWQLGFLIQVSLIIMLLSSMLFIDKPHNWSCMAGQVTLALGFSLCLSCLLGKTSSLFLAYRISKSKTQLTSMHPLYRKIIVLISVLAEIGICTAYLILEPPMVYKNMESQNTKIILGCNEISIEFLYSMFGIDAFLALLCFLTTFVARQLPDNYYEGKCITFGMLVFFIIWMSFVPVYLSTKGKFKMAVEIFAILASSHGLLGCIFAPKCLIILLRPERNTSEIVCGRVSTTDNCIQLTSAFVSSELNNTTVSTVLDDRVLIYMCPLKLQ

SEQ ID NO: 6 - DNA, 1140 bp, Artificial Sequence
  This degenerate nucleotide sequence encodes the amino acid sequence of SEQ ID NO5.
ytnccncaywsngtntgyacngaygtntgyccnccnggnacnggnmgnggnttygtncarmgngarccnathtgytgyttygaywsnathccntgygcngayggncaygtnwsnmgnaarccnggngarmgngartgygarcartgyggngargaytaytggwsnaaygcncaraarwsngartgygtnytnaargargtngartayytngcntaygaygargcnytnggnttyacnytngtnathytnwsngtnttyggngcnttygtngtnytngcngtnacngcngtntaygtnathcaymgncayacnccnytngtnaaygcnwsngaytggcarytnggnttyytnathcargtnwsnytnathathatgytnytnwsnwsnatgytnttyathgayaarccncayaaytggwsntgyatggcnggncargtnacnytngcnytnggnttywsnytntgyytnwsntgyytnytnggnaaracnwsnwsnytnttyytngcntaymgnathwsnaarwsnaaracncarytnacnwsnatgcayccnytntaymgnaarathathgtnytnathwsngtnytngcngarathggnathtgyacngcntayytnathytngarccnccnatggtntayaaraayatggarwsncaraayacnaarathathytnggntgyaaygarathwsnathgarttyytntaywsnatgttyggnathgaygcnttyytngcnytnytntgyttyytnacnacnttygtngcnmgncarytnccngayaaytaytaygarggnaartgyathacnttyggnatgytngtnttyttyathathtggatgwsnttygtnccngtntayytnwsnacnaarggnaarttyaaratggcngtngarathttygcnathytngcnwsnwsncayggnytnytnggntgyathttygcnccnaartgyytnathathytnytnmgnccngarmgnaayacnwsngarathgtntgyggnmgngtnwsnacnacngayaaytgyathcarytnacnwsngcnttygtnwsnwsngarytnaayaayacnacngtnwsnacngtnytngaygaymgngtnytnathtayatgtgyccnytnaarytncar

SEQ ID NO: 7 - DNA, 2781 bp, Artificial Sequence
  Chimeric receptor.
atgtttgagaggcgcaaagagcaagacgagggaccaggaatccatgaatttcttgcatttttatgggctgaattgggctctgaagccaaagaagagaaagaagaagaacggacctgccggttgctgggcaagtgtgtagatgccgaaaaccattcccttgttattggaggactgtttcctattgactccaggaccatcccagcaaatgagtctattttggagccagcatcagcaaaatgtgaagggtttaactttcagagattccgctggatgaaagccatgatccacatgatcaaggagattaataagaggaaggatattttgcccaacatcactctgggctatcagatctttgatacctgttttaccatctccaaatcagtggaagcagtcttggtatttcttacagggcaggaagaaaacaggcccaattttagaaacagcactggagcatttccggcaggaattgttggagcaggtggatcattcttatcagttcctgcttcaagaattctagggttatattatttgcctcaggtgggctatacctctacctgcgtgattcttagtgacaaataccagtttccatcttatcttcgtgtaatagccagcgataagatccagtcgaaggctgtggtaaaacgtatccaacactttggttgggtctgggtaggtgctattgcagctgatgatgattatgggaaatatggagtaaaaacttttaaggaaaaaatggagagtgccaacctctgtgttgctttctctgaaaccattcccaaagtctactccaatgagaaaatgcagaaggctgttaaggcagtaaagacttccactgccaaagtcattgtgctttatacttctgacattgacctcagcctgtttgtgctggaaatgattcatcataacataactgacaggacatggatagccaccgaagcctggattacctctgctctcattgcaaagcctgagtatttcccctattttggtggaactattggatttgcaacaccaagaagtgttataccaggactaaaagaatttctttatgatgtacaccctaacaaggatccaaatgatgtcttgaccattgaattctggcaaactgcttttaactgtacctggcccaacagcagtgttccttataacgtggatcacagagtgaatatgactgggaaagaagacagactgtatgacatgtctgatcagctctgcactggagaggagaagctggaagatctgaaaaacacctatctggatacatcacagctaagaattacaaaacaatgtaaacaagctgtatatgctatagctcatggcctggatcatctcagcagatgtcaagaagggcagggaccatttggctcaaatcagcaatgtgcatatatacctacctttgatttctggcagctaatgtactatatgaaagaaattaaatttaaatcacatgaggataaatgggtaattctggatgataatggagatttgaaaaatggacactatgatgtcctaaactggcacttagatgatgagggagaaatttcctttgtgacagttgggagatttaactttagatctacaaactttgagcttgttattccaacgaattctacaatattttggaacactgagtcatcaaggcttccccattcagtgtgtactgatgtgtgtcctcctgggactggaaggggattcgttcagagggaaccaatatgctgctttgactccatcccatgtgctgatggacacgtgtcacggaaaccaggtgaaagggagtgtgaacaatgtggtgaagactattggtcaaatgcacaaaagagcgagtgtgtgctgaaagaggtggaataccttgcttatgatgaggccctgggattcacacttgtcattctttctgtctttggggcatttgtggtcttggcagtcacagctgtgtatgtgatacacaggcacactcccctggtgaacgccagtgactggcagctgggctttctcattcaggtttctctgatcatcatgctgctgtcgtccatgcttttcattgacaagccacacaactggtcctgcatggctggccaggtcactctggcactgggcttttctctttgcctgtcttgccttcttggaaagactagttcactgtttttagcctacagaatttccaaatccaaaactcaacttacatccatgcaccccctttatcggaaaatcattgtgctaatctctgttctagcggagattggcatatgtacagcctacttgatattggaacctcccatggtatacaagaacatggaatctcaaaatacaaagatcattctgggatgcaatgaaatttccatagagtttttgtactcgatgtttggaattgatgccttcttagccttgctatgctttcttacaacttttgtggctcgccagttaccagataattactatgaaggaaaatgcatcacctttgggatgcttgtctttttcatcatttggatgtcttttgtccctgtttatttgagcaccaaaggcaagttcaaaatggctgtggaaatatttgcaatcttggcatccagccatggcttgttgggttgtatatttgctcctaagtgcctcattattttgctgaggccagagaggaacaccagtgaaattgtttgtggaagagtctccaccacagataattgcatccaactgacctcagcttttgtgagcagtgagcttaacaataccacagtgtcaactgttctggatgacagagttttgatttacatgtgtcctttgaagctgcaa

SEQ ID NO: 8 - protein, 927 aa, Artificial Sequence
  Chimeric receptor.
MFERRKEQDEGPGIHEFLAFLWAELGSEAKEEKEEERTCRLLGKCVDAENHSLVIGGLFPIDSRTIPANESILEPASAKCEGFNFQRFRWMKAMIHMIKEINKRKDILPNITLGYQIFDTCFTISKSVEAVLVFLTGQEENRPNFRNSTGAFPAGIVGAGGSFLSVPASRILGLYYLPQVGYTSTCVILSDKYQFPSYLRVIASDKIQSKAVVKRIQHFGWVWVGAIAADDDYGKYGVKTFKEKMESANLCVAFSETIPKVYSNEKMQKAVKAVKTSTAKVIVLYTSDIDLSLFVLEMIHHNITDRTWIATEAWITSALIAKPEYFPYFGGTIGFATPRSVIPGLKEFLYDVHPNKDPNDVLTIEFWQTAFNCTWPNSSVPYNVDHRVNMTGKEDRLYDMSDQLCTGEEKLEDLKNTYLDTSQLRITKQCKQAVYAIAHGLDHLSRCQEGQGPFGSNQQCAYIPTFDFWQLMYYMKEIKFKSHEDKWVILDDNGDLKNGHYDVLNWHLDDEGEISFVTVGRFNFRSTNFELVIPTNSTIFWNTESSRLPHSVCTDVCPPGTGRGFVQREPICCFDSIPCADGHVSRKPGERECEQCGEDYWSNAQKSECVLKEVEYLAYDEALGFTLVILSVFGAFVVLAVTAVYVIHRHTPLVNASDWQLGFLIQVSLIIMLLSSMLFIDKPHNWSCMAGQVTLALGFSLCLSCLLGKTSSLFLAYRISKSKTQLTSMHPLYRKIIVLISVLAEIGICTAYLILEPPMVYKNMESQNTKIILGCNEISIEFLYSMFGIDAFLALLCFLTTFVARQLPDNYYEGKCITFGMLVFFIIWMSFVPVYLSTKGKFKMAVEIFAILASSHGLLGCIFAPKCLIILLRPERNTSEIVCGRVSTTDNCIQLTSAFVSSELNNTTVSTVLDDRVLIYMCPLKLQ

SEQ ID NO: 9 - DNA, 2781 bp, Artificial Sequence
  This degenerate nucleotide sequence encodes the amino acid sequence of SEQ ID8.
atgttygarmgnmgnaargarcargaygarggnccnggnathcaygarttyytngcnttyytntgggcngarytnggnwsngargcnaargargaraargargargarmgnacntgymgnytnytnggnaartgygtngaygcngaraaycaywsnytngtnathggnggnytnttyccnathgaywsnmgnacnathccngcnaaygarwsnathytngarccngcnwsngcnaartgygarggnttyaayttycarmgnttymgntggatgaargcnatgathcayatgathaargarathaayaarmgnaargayathytnccnaayathacnytnggntaycarathttygayacntgyttyacnathwsnaarwsngtngargcngtnytngtnttyytnacnggncargargaraaymgnccnaayttymgnaaywsnacnggngcnttyccngcnggnathgtnggngcnggnggnwsnttyytnwsngtnccngcnwsnmgnathytnggnytntaytayytnccncargtnggntayacnwsnacntgygtnathytnwsngayaartaycarttyccnwsntayytnmgngtnathgcnwsngayaarathcarwsnaargcngtngtnaarmgnathcarcayttyggntgggtntgggtnggngcnathgcngcngaygaygaytayggnaartayggngtnaaracnttyaargaraaratggarwsngcnaayytntgygtngcnttywsngaracnathccnaargtntaywsnaaygaraaratgcaraargcngtnaargcngtnaaracnwsnacngcnaargtnathgtnytntayacnwsngayathgayytnwsnytnttygtnytngaratgathcaycayaayathacngaymgnacntggathgcnacngargcntggathacnwsngcnytnathgcnaarccngartayttyccntayttyggnggnacnathggnttygcnacnccnmgnwsngtnathccnggnytnaargarttyytntaygaygtncayccnaayaargayccnaaygaygtnytnacnathgarttytggcaracngcnttyaaytgyacntggccnaaywsnwsngtnccntayaaygtngaycaymgngtnaayatgacnggnaargargaymgnytntaygayatgwsngaycarytntgyacnggngargaraarytngargayytnaaraayacntayytngayacnwsncarytnmgnathacnaarcartgyaarcargcngtntaygcnathgcncayggnytngaycayytnwsnmgntgycargarggncarggnccnttyggnwsnaaycarcartgygcntayathccnacnttygayttytggcarytnatgtaytayatgaargarathaarttyaarwsncaygargayaartgggtnathytngaygayaayggngayytnaaraayggncaytaygaygtnytnaaytggcayytngaygaygarggngarathwsnttygtnacngtnggnmgnttyaayttymgnwsnacnaayttygarytngtnathccnacnaaywsnacnathttytggaayacngarwsnwsnmgnytnccncaywsngtntgyacngaygtntgyccnccnggnacnggnmgnggnttygtncarmgngarccnathtgytgyttygaywsnathccntgygcngayggncaygtnwsnmgnaarccnggngarmgngartgygarcartgyggngargaytaytggwsnaaygcncaraarwsngartgygtnytnaargargtngartayytngcntaygaygargcnytnggnttyacnytngtnathytnwsngtnttyggngcnttygtngtnytngcngtnacngcngtntaygtnathcaymgncayacnccnytngtnaaygcnwsngaytggcarytnggnttyytnathcargtnwsnytnathathatgytnytnwsnwsnatgytnttyathgayaarccncayaaytggwsntgyatggcnggncargtnacnytngcnytnggnttywsnytntgyytnwsntgyytnytnggnaaracnwsnwsnytnttyytngcntaymgnathwsnaarwsnaaracncarytnacnwsnatgcayccnytntaymgnaarathathgtnytnathwsngtnytngcngarathggnathtgyacngcntayytnathytngarccnccnatggtntayaaraayatggarwsncaraayacnaarathathytnggntgyaaygarathwsnathgarttyytntaywsnatgttyggnathgaygcnttyytngcnytnytntgyttyytnacnacnttygtngcnmgncarytnccngayaaytaytaygarggnaartgyathacnttyggnatgytngtnttyttyathathtggatgwsnttygtnccngtntayytnwsnacnaarggnaarttyaaratggcngtngarathttygcnathytngcnwsnwsncayggnytnytnggntgyathttygcnccnaartgyytnathathytnytnmgnccngarmgnaayacnwsngarathgtntgyggnmgngtnwsnacnacngayaaytgyathcarytnacnwsngcnttygtnwsnwsngarytnaayaayacnacngtnwsnacngtnytngaygaymgngtnytnathtayatgtgyccnytnaarytncar

SEQ ID NO: 10 - protein, 16 aa, Artificial Sequence
  Peptide linker.
GGSGGSGGGGSGGGGS

(19) United States
(12) Patent Application Publication
(10) Pub. No.: US 2002/0146418 A1
(43) Pub. Date: Oct. 10, 2002

(54) Human V2 vomeronasal receptor

(76) Inventors: Si Lok, Seattle, WA (US); James L. Holloway, Seattle, WA (US)

Correspondence Address: Phillip B.C. Jones, J.D., Ph.D., ZymoGenetics, Inc., 1201 Eastlake Avenue East, Seattle, WA, 98102, US

(21) Appl. No.: 10/003,356

(22) Filed: Nov. 15, 2001

Related U.S. Application Data
(60) Provisional application No. 60/252,373, filed on Nov. 21, 2000.

(51) Int. Cl. A61K 39/395; G01N 33/53; C07H 21/04; C12P 21/02; C12N 5/06; C07K 14/705

(52) U.S. Cl. 424/143.1; 435/69.1; 435/325; 435/320.1; 530/350; 536/23.2; 435/7.1; 530/388.22

(57) ABSTRACT

In mammals, the vomeronasal organ, which detects phermones, resides in a blind-ended pouch within the septum of the nose. Vomeronasal organ-derived signals bypass higher cognitive centers and are processed directly in regions of the amygdala and hypothalamus, which have been implicated in the regulation of innate behavior, reproductive physiology, and other neuroendocrine responses. Zvn2R1 encodes a human vomeronasal receptor.

### CROSS-REFERENCE TO RELATED APPLICATIONS

This application claims the benefit of U.S. Provisional application No. 60/252,373 (filed Nov. 21, 2000), the contents of which are incorporated by reference.

### TECHNICAL FIELD

The present invention relates generally to a new protein expressed by human cells. In particular, the present invention relates to a novel gene that encodes a receptor, designated as “Zvn2R1,” and to nucleic acid molecules encoding Zvn2R1 polypeptides.

### BACKGROUND OF THE INVENTION

Olfaction is an ancient sense, rudiments of which can be found in the most primitive single-celled organisms (see generally, Tirindelli et al., TINS 11:482 (1998); Keveme, Science 286:716 (1999): Liman, Current Opinion in Neurobiology 6:487 (1996); Buck, Cell 65:175 (2000)). The importance of this sense is exemplified by the fact that humans are capable of perceiving thousands of discrete odors, and that more than 1% of the genes in the human genome are devoted to olfaction. Olfaction has an aesthetic component that is capable of invoking emotion and memory leading to measured thoughts and response to the everyday environment. However, in some species, a diverse class of molecules, generally referred to as pheromones, can elicit innate and stereotyped behaviors that are likely to result from non-conscious perception.

At present, the majority of the identified pheromones are from insects. Many insect species produce potent volatile chemical compounds, which attract potential mates over long distances (Kaissling, Ann. Rev. Neurosci. 9:121 (1986); Masson and Mustaparta, Physiol. Rev. 70:199 (1990)). Synthetic versions of certain pheromones are used as chemo-attractants to control insect pests. Members of the animal kingdom are also known to produce chemical pheromones for intra-species communication. F-prostaglandins and steroids, for example, have been shown to induce sperm production and mating in fish (Stacey and Sorensen, Can. J. Zool. 64:2412 (1986); Sorensen et al., Biol. Reprod. 39:1039 (1988)). In reptiles, a family of dianeackerone-related steroidal esters was characterized from the crocodilian paracloacal gland secretions, which are thought to contain pheromones that may play a role in nesting and mating activities (Whyte et al., Proc. Nat'l Acad. Sci. (USA) 96:12246 (1999); Yang et al., Proc. Nat'l Acad. Sci. (USA) 96:12251 (1999)). A series of nonvolatile saturated and monosaturated long-chain methyl ketones, and compounds containing squalene were shown to induce courtship behavior in garter snakes (Mason et al., Science 293:290 (1989)). Recently, a proteinaceous pheromone affecting female receptivity was isolated from a terrestrial salamander (Rollmann et al., Science 285:1907 (1999)), and a peptide with female-attracting activity was identified in newts (Kikuyama et al., Science 267:1643 (1995)).

Mammalian pheromones have also been described. For example, studies indicate that newborn rabbits, rats, and pigs are directed to the nipple by a pheromone produced by the nipple or by the surrounding areola region (Blass and Teicher, Science 210:15 (1980); Keil et al., Physiol. Behav. 47:525 (1990); Morrow-Tesch and McGlone, J. Anim. Sci. 68:3563 (1990)). Various phermones affect sexual maturation and behavior, as well. As an illustration, 6-hydroxy-6-methyl-3-heptanone is a pheromone that accelerates puberty in female mice (Novotny et al., Chemistry & Biology 6:377 (1999)). Two major volatile constituents of the male rodent preputial gland, E,E-α-farnesene and E-β-famesene were shown to attract females, and to induce estrus (Jemiolo et al., Physiology & Behavior 50:1119 (1991); Ma et al., Chem. Senses 24:289 (1991)). Thiazole (2-sec-butyl-4,5-dihydrothiazole) and brevicomin (2,3-dehydro-exo-brevicomin), volatile chemicals found in rodent urine, serve as attractants for females, inducers of estrous, and stimulators of inter-male aggression (Jemiolo et al., Anim. Behav. 33:1114 (1985); Jemiolo et al., Proc. Nat'l Acad. Sci. (USA) 83:4576 (1986); Novotny et al., Proc. R. Soc. Lond. B. Biol. Sci. 266:2017 (2000)).

The existence of human pheromones was first suggested by the observation that women living together can develop synchronized menstrual cycles under specific conditions (McClintock, Nature 291:244 (1971)). The causal agents were later attributed to odorless pheromone-like substances produced in female underarms (Stern and McClintock, Nature 392:177 (1998)). Some studies suggest that androsterone, when administered nasally, may alter human social behavior (Gustavson et al., Psychol. 101:210 (1987); Filsinger et al., J. Comp. Psychol. 98:219 (1984); Cowley and Brooksbank, J. Steroid Biochem. Molec. Biol. 39:647 (1991); Gower and Ruparelia, J. Endocrinol. 137:167 (1993); Pause et al., Physiology & Behavior 68:129 (1999); Grosser et al., Psychoneuroendocrinology 25:289 (2000)). Moreover, human infants, like other mammalian infants, are apparently responsive to olfactory cues emanating from their mother's nipple or areola region (Winberg and Porter, Acta Paediatr. 87:6 (1998); Porter and Winberg, Neuroscience and Biobehavioral Reviews 23:439 (1999)).

In mammals, the main olfactory system resides in the sensory epithelium within the posterior recess of the nasal cavity. In contrast, the vomeronasal organ, which detects phermones, resides relatively anterior, in a blind-ended pouch within the septum of the nose (Stensaas et al., J. Steroid. Biochem. Molec. Biol. 39:553 (1991); Monti-Bloch et al., Ann. N.Y. Acad. Sci. 30:373 (1998)). In rodents, the vomeronasal organ is a cartilage-encased tubular structure located at the base of the nasal septum, which opens into the nasal cavity via a single duct. Ordorants dissolved in the nasal cavity are transported into the vomeronasal organ by changes in the blood volume. Like the main olfactory epithelium, which detects everyday odorants and perhaps some pheromones, the neuroepithelium of the vomeronasal organ contains sensory neurons that project axons to the brain (Belluscio et al., Cell 97:209 (1999); Rodriguez et al., Cell 97:199 (1999)). However, the signals derived from the main olfactory epithelium and the vomeronasal organ are distinct and remain separate at all levels of the nervous system. Sensory input from the main olfactory epithelium ultimately reaches multiple regions of the brain, including the frontal cortex, which is believed to process the conscious perception of odors. Vomeronasal organ-derived signals, however, bypass higher cognitive centers and are processed directly in regions of the amygdala and hypothalamus, which have been implicated in the regulation of innate behavior, reproductive physiology, and other neuroendocrine responses.

A family of putative rat pheromone receptors, VI, was discovered in 1995 (Dulac and Axel, Cell 83:195 (1995); Dulac and Axel, Chem. Senses 23:467 (1998)). This discovery was followed by the identification of a second class of putative rodent pheromone receptors, V2 (Ryba and Trindelli, Neuron 19:371 (1997); Herrada and Dulac, Cell 90:763 (1997); Matsunami and Buck, Cell 90:775 (1997)). Sequence analysis indicates that the V1 and V2 receptor genes comprise two novel families of seven-transmembrane domain G-protein coupled proteins, which are distinct from the odorant receptors expressed in the main olfactory epithelium, or to other families of seven-transmembrane domain receptors (Buck and Axel, Cell 65:175 (1991)). Southern blot analysis indicated that the rat V1 family includes approximately 35 members, each of which is expressed in a small subpopulation of vomeronasal organ neurons, whereas the rat V2 receptor family contains about 150 members.

The V2 receptors are structurally related to the metabotropic glutamate receptors, which have large N-terminal domains that can independently bind ligand (O'Hara et al., Neuron 11:41 (1993); Okamoto et al., J. Biol. Chem. 272:13089 (1998); Han and Hampson, J. Biol. Chem. 274:10008 (1999)). The metabotropic glutamate receptor ligand binding domain and that of the related calcium receptor apparently bind their cognate ligands as a homo-dimer of two receptor subunits (Okamoto et al., J. Biol. Chem. 272:13089 (1998); Han and Hampson, J. Biol. Chem. 274:10008 (1999); Goldsmith et al., J. Biol. Chem. 274:11303 (1999)), suggesting that the N-terminal domain of the V2 receptors bind their ligands in a similar configuration. In contrast to the V2 receptors, the short N-terminal domains of the V1 receptors suggest that a receptor ligand binding pocket may form from the transmembrane segments or the peptide loops between transmembrane segments. Although the different structure of the V1 and V2 receptors ligand-binding pockets suggest that they recognize different types of ligands, no ligands for these classes of receptors have yet been identified.

The V1 and V2 signaling pathways appear to be different from that of the main olfactory system. The V1 receptors are coupled to Gαi2, while the V2 receptors are coupled to Gα0. The well characterized olfactory transduction system involves activation of adenylyl cyclase III through Gαolf, resulting in a rise in cAMP levels and the subsequent opening of a dimeric subunit cyclic-nucleotide-gated channel (Firestein, Current Opinion in Neurobiology 2:444 (1992); Reed, Neuron 8:205 (1992); Brunet et al. Neuron 17:681 (1996); Belluscio et al., Cell 97:209 (1999)). Gαolf and one subunit of the cyclic-nucleotide-gated channel appear to be absent in the vomeronasal organ neurons, indicating that there is an alternative pathway for sensory transduction in the vomeronasal organ (Berghard et al., Proc. Nat'l Acad. Sci. (USA) 93:2365 (1996); Berghard and Buck, J. Neurosci. 16:909 (1996)). This suggestion is supported by the recent finding that the main signaling pathway of aphrodisin, a hamster sex pheromone, activates the inositol triphosphate pathway in the vomeronasal organ with no observed increase in intracellular cAMP level, and the blockage of responsiveness to the major urinary proteins and their ligands by inhibitors to inositol triphosphate-mediated pathway in rat vomeronasal sensory neurons (Inamura et al., Neurosci. Lett. 233:129 (1997); Kroner et al., NeuroReport 7:2989 (2000)).

A recent human anatomical study failed to locate the vomeronasal organ in a significant numbers of adult volunteers, and failed to identify neurons in vomeronasal nerve bundles by using a wide variety of neural markers (Eloit et al., Congress of the European Chemoreception Research Organization, Sienna, Italy, September 8 to 13, 1998 (Abstract 148)). In contrast to these findings, electron microscopy of adult human vomeronasal organ shows the presence of microvillar cells and unmyelinated intraepithelial axons in the pseudostratified epithelial lining indicating the presence of a functional chemosensory system (Stensaas et al., J. Steroid. Biochem. Molec. Biol. 39:553 (1991); Jahnke and Merker, Am. J. Rhinol. 14:63 (2000)). Brain imaging studies also reveal consistent activation of the hypothalamus, amygdala and cingulate gyrus-related structure during stimulation which are supportive of a functional vomeronasal organ system in adult human (Monti-Bloch et al., Ann. N.Y. Acad. Sci. 30:373 (1998)). Moreover, studies in which chemosensory substances were administered to the vomeronasal organ of adults indicate that the vomeronasal organ is a functional chemosensory organ with sexually dimorphic specificity and the ability to transduce signals to mediate behavioral and electrophysiological effects of phermones (Mont-Bloch et al., Psychoneuroendocrinology 19:673 (1994); Grosser et al., Psychoneuroendocrinology 25:289 (2000); Berliner, U.S. Pat. No. 5,278,141; Jennings-White et al., U.S. Pat. No. 5,962,443).

To date, only pseudogenes have been identified for the human V2 vomeronasal organ receptor genes (Keverne, Science 286:716 (1999)). With the exception of these pseudogenes, human orthologs of the rodent V2 receptors have not been isolated (Keveme, Science 286:716 (1999)). A need, therefore, exists for the identification of genes that encode these functional human vomeronasal organ receptors.

### SUMMARY OF THE INVENTION

The present invention provides a novel human V2 receptor, designated “Zvn2R1.” The present invention also provides Zvn2R1 polypeptides and Zvn2R1 fusion proteins, as well as nucleic acid molecules encoding such polypeptides and proteins, and methods for using these nucleic acid molecules and amino acid sequences.

### DETAILED DESCRIPTION OF THE INVENTION

1. Overview

Two portions of a human Zvn2R1 receptor were identified. The N-termiinus of the receptor has the following amino acid sequence: MFERRKEQDE GPGIHEFLAF LWAELGSEAK EEKEEERTCR LLGKCVDAEN HSLVIGGLFP IDSRTIPANE SILEPASAKC EGFNFQRFRW MKAMIHMIKE INKRKDILPN ITLGYQIFDT CFTISKSVEA VLVFLTGQEE NRPNFRNSTG AFPAGIVGAG GSFLSVPASR ILGLYYLPQV GYTSTCVILS DKYQFPSYLR VIASDKIQSK AVVKRIQHF (SEQ ID NO:2). The C-terminus of the receptor has the following amino acid sequence:LPHSVCTDVC PPGTGRGFVQ REPICCFDSI PCADGHVSRK PGERECEQCG EDYWSNAQKS ECVLKEVEYL AYDEALGFTL VILSVFGAFV VLAVTAVYVI HRHTPLVNAS DWQLGFLIQV SLIIMLLSSM LFIDKPHNWS CMAGQVTLAL GFSLCLSCLL GKTSSLFLAY RISKSKTQLT SMHPLYRKII VLISVLAEIG ICTAYLILEP PMVYKNMESQ NTKIILGCNE ISIEFLYSMF GIDAFLALLC FLTTFVARQL PDNYYEGKCI TFGMLVFFII WMSFVPVYLS TKGKFKMAVE IFAILASSHG LLGCIFAPKC LIILLRPERN TSEIVCGRVS TTDNCIQLTS AFVSSELNNT TVSTVLDDRV LIYMCPLKLQ (SEQ ID NO:5). Illustrative nucleotide sequences that encode the amino acid sequences of SEQ ID NO:2 and SEQ ID NO:5 are provided by SEQ ID NO: 1 and SEQ ID NO:4, respectively.

A chimeric receptor was designed by aligning these human sequences and the sequence of the murine tissue-type vomeronasal putative pheromone receptor V2R2 (GenBank Accession No. AF053986). The nucleotide and amino acid sequences of the chimeric receptor are provided by SEQ ID NO:7 and SEQ ID NO:8, respectively. Table 1 provides structural features of the chimeric Zvn2R1 receptor and the human polypeptides.

TABLE 1

| Domain | Amino Acid Sequence Location |
|---|---|
| Signal sequence | 1 - 29 of SEQ ID NOs:2 and 8 |
| Extracellular domain | 1 - 621 of SEQ ID NO:8 |
| Ligand binding domain | 30 - 610 of SEQ ID NO:8 |
| Transmembrane domain-1 | 622 - 647 of SEQ ID NO:8; 75 - 100 of SEQ ID NO:5 |
| Intracellular domain | 648 - 660 of SEQ ID NO:8; 101 - 113 of SEQ ID NO:5 |
| Transmembrane domain-2 | 661 - 681 of SEQ ID NO:8; 114 - 134 of SEQ ID NO:5 |
| Extracellular domain | 682 - 692 of SEQ ID NO:8; 135 - 145 of SEQ ID NO:5 |
| Transmembrane domain-3 | 693 - 717 of SEQ ID NO:8; 146 - 170 of SEQ ID NO:5 |
| Intracellular domain | 718 - 735 of SEQ ID NO:8; 171 - 188 of SEQ ID NO:5 |
| Transmembrane domain-4 | 736 - 755 of SEQ ID NO:8; 189 - 208 of SEQ ID NO:5 |
| Extracellular domain | 756 - 777 of SEQ ID NO:8; 209 - 230 of SEQ ID NO:5 |
| Transmembrane domain-5 | 778 - 802 of SEQ ID NO:8; 231 - 255 of SEQ ID NO:5 |
| Intracellular domain | 803 - 815 of SEQ ID NO:8; 256 - 268 of SEQ ID NO:5 |
| Transmembrane domain-6 | 816 - 836 of SEQ ID NO:8; 269 - 289 of SEQ ID NO:5 |
| Extracellular domain | 837 - 847 of SEQ ID NO:8; 290 - 330 of SEQ ID NO:5 |
| Transmembrane domain-7 | 848 - 872 of SEQ ID NO:8; 331 - 325 of SEQ ID NO:5 |
| Intracellular domain | 873 - 927 of SEQ ID NO:8; 326 - 380 of SEQ ID NO:5 |

As described below, the present invention provides isolated polypeptides comprising an amino acid sequence that is at least 70%, at least 80%, at least 90%, or at least 95% identical to the amino acid sequence of SEQ ID NOs:2 or 5. Certain of these polypeptides can specifically bind with an antibody that specifically binds with a polypeptide consisting of the amino acid sequence of SEQ ID NOs:2 or 5. Illustrative polypeptides include a polypeptide comprising the amino acid sequence of SEQ ID NOs:2 or 5.

The present invention also includes variant Zvn2R1 polypeptides, wherein the amino acid sequence of the variant polypeptide shares an identity with the amino acid sequence of SEQ ID NOs:2 or 5 selected from the group consisting of at least 70% identity, at least 80% identity, at least 90% identity, at least 95% identity, or greater than 95% identity, and wherein any difference between the amino acid sequence of the variant polypeptide and the amino acid sequence of SEQ If) NOs:2 or 5 is due to one or more conservative amino acid substitutions.

The present invention further provides polypeptides comprising, or consisting of, the following amino acid sequences: the amino acid sequence of SEQ ID NO:8, amino acid residues 1 to 621 of SEQ ID NO:8, amino acid residues 30 to 621 of SEQ ID NO:8, amino acid residues 1 to 610 of SEQ ID NO:8, and amino acid residues 30to 610of SEQ ID NO:8.

The present invention further provides antibodies and antibody fragments that specifically bind with such polypeptides. For example, the present invention includes an antibody or antibody fragment that specifically binds with an amino acid sequence disclosed herein. Exemplary antibodies include polyclonal antibodies, murine monoclonal antibodies, humanized antibodies derived from murine monoclonal antibodies, and human monoclonal antibodies. Illustrative antibody fragments include F(ab′)₂, F(ab)₂, Fab′, Fab, Fv, scFv, and minimal recognition units. The present invention further provides compositions comprising a carrier and a peptide, polypeptide, antibody, or anti-idiotype antibody described herein.

The present invention also provides isolated nucleic acid molecules that encode a Zvn2R1 polypeptide, wherein the nucleic acid molecule is selected from the group consisting of: (a) a nucleic acid molecule comprising the nucleotide sequence of SEQ ID NOs:3 or 6, (b) a nucleic acid molecule encoding an amino acid sequence that comprises the amino acid sequence of SEQ ID NOs:2 or 5, and (c) a nucleic acid molecule that remains hybridized following stringent wash conditions to a nucleic acid molecule comprising a nucleotide sequence selected from the group consisting of: the nucleotide sequence of SEQ ID NOs:1 or 4, or the complement of the nucleotide sequence of SEQ ID NOs: 1 or 4. Illustrative nucleic acid molecules include those in which any difference between the amino acid sequence encoded by nucleic acid molecule (c) and the corresponding amino acid sequence of SEQ ID NOs:2 or 5 is due to a conservative amino acid substitution. The present invention further contemplates isolated nucleic acid molecules that comprise the nucleotide sequence of SEQ ID NOs: 1, 4, or 7.

The present invention also includes vectors and expression vectors comprising such nucleic acid molecules. Such expression vectors may comprise a transcription promoter, and a transcription terminator, wherein the promoter is operably linked with the nucleic acid molecule, and wherein the nucleic acid molecule is operably linked with the transcription terminator. An illustrative expression vector comprises the nucleotide sequence of nucleotides 88 to 1830 of SEQ ID NO:7, a transcription promoter, and a transcription terminator, wherein the promoter is operably linked with the nucleotide sequence, and wherein the nucleotide sequence is operably linked with the transcription terminator.

The present invention further includes recombinant host cells and recombinant viruses comprising these vectors and expression vectors. Illustrative host cells include bacterial, yeast, avian, fungal, insect, mammalian, and plant cells. Recombinant host cells comprising such expression vectors can be used to produce Zvn2R1 polypeptides by culturing such recombinant host cells that comprise the expression vector and that produce the Zvn2R1 protein, and, optionally, isolating the Zvn2R1 protein from the cultured recombinant host cells. The present invention also includes the products of such processes.

In addition, the present invention provides pharmaceutical compositions comprising a pharmaceutically acceptable carrier and at least one of such an expression vector or recombinant virus comprising such expression vectors. The present invention further includes pharmaceutical compositions, comprising a pharmaceutically acceptable carrier and a polypeptide described herein.

The present invention also contemplates methods for detecting the presence of Zvn2R1 RNA in a biological sample, comprising the steps of (a) contacting a Zvn2R1 nucleic acid probe under hybridizing conditions with either (i) test RNA molecules isolated from the biological sample, or (ii) nucleic acid molecules synthesized from the isolated RNA molecules, wherein the probe has a nucleotide sequence comprising a portion of the nucleotide sequence of SEQ ID NO:1s or 4, or their complements, and (b) detecting the formation of hybrids of the nucleic acid probe and either the test RNA molecules or the synthesized nucleic acid molecules, wherein the presence of the hybrids indicates the presence of Zvn2R1 RNA in the biological sample.

The present invention further provides methods for detecting the presence of Zvn2R1 polypeptide in a biological sample, comprising the steps of. (a) contacting the biological sample with an antibody or an antibody fragment that specifically binds with a polypeptide consisting of the amino acid sequence of SEQ ID NOs:2 or 5, wherein the contacting is performed under conditions that allow the binding of the antibody or antibody fragment to the biological sample, and (b) detecting any of the bound antibody or bound antibody fragment. Such an antibody or antibody fragment may further comprise a detectable label selected from the group consisting of radioisotope, fluorescent label, chemiluminescent label, enzyme label, bioluminescent label, and colloidal gold.

The present invention also provides kits for performing these detection methods. For example, a kit for detection of Zvn2R1 gene expression may comprise a container that comprises a nucleic acid molecule, wherein the nucleic acid molecule is selected from the group consisting of (a) a nucleic acid molecule comprising the nucleotide sequence of SEQ ID NOs:1 or 4, (b) a nucleic acid molecule comprising the complement of the nucleotide sequence of SEQ ID NOs:1 or 4, (c) a nucleic acid molecule that is a fragment of (a) consisting of at least eight nucleotides, and (d) a nucleic acid molecule that is a fragment of (b) consisting of at least eight nucleotides. Such a kit may also comprise a second container that comprises one or more reagents capable of indicating the presence of the nucleic acid molecule. On the other hand, a kit for detection of Zvn2R1 protein may comprise a container that comprises an antibody, or an antibody fragment, that specifically binds with a polypeptide consisting of the amino acid sequence of SEQ ID NOs:2 or 5.

The present invention also contemplates anti-idiotype antibodies, or anti-idiotype antibody fragments, that specifically bind an antibody or antibody fragment that specifically binds a polypeptide consisting of the amino acid sequence of SEQ ID NO:2, the amino acid sequence of SEQ ID NO:5, or a polypeptide consisting of the amino acid sequence of amino acid residues 30 to 610 of SEQ ID NO:8..

The present invention also contemplates methods for detecting a ligand of Zvn2R1 within a test sample, comprising the steps of (a) contacting the test sample with a polypeptide that comprises the amino acid sequence of SEQ ID NOs:2, 5, or 8 and (b) detecting the binding of the polypeptide to ligand in the sample. Such a polypeptide can be membrane bound within a cultured cell, and the detecting step would comprise measuring a biological response in the cultured cell. In another variation of these methods, the source of a Zvn2R1 polypeptide is a cell membrane preparation obtained from cells that produce Zvn2R1.

These and other aspects of the invention will become evident upon reference to the following detailed description. In addition, various references are identified below and are incorporated by reference in their entirety.

2. Definitions In the description that follows, a number of terms are used extensively. The following definitions are provided to facilitate understanding of the invention.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “complement of a nucleic acid molecule” refers to a nucleic acid molecule having a complementary nucleotide sequence and reverse orientation as compared to a reference nucleotide sequence. For example, the sequence 5′ ATGCACGGG 3′ is complementary to 5′ CCCGTGCAT 3′.

The term “contig” denotes a nucleic acid molecule that has a contiguous stretch of identical or complementary sequence to another nucleic acid molecule. Contiguous sequences are said to “overlap” a given stretch of a nucleic acid molecule either in their entirety or along a partial stretch of the nucleic acid molecule.

The term “degenerate nucleotide sequence” denotes a sequence of nucleotides that includes one or more degenerate codons as compared to a reference nucleic acid molecule that encodes a polypeptide. Degenerate codons contain different triplets of nucleotides, but encode the same amino acid residue (i.e., GAU and GAC triplets each encode Asp).

The term “structural gene” refers to a nucleic acid molecule that is transcribed into messenger RNA (mRNA), which is then translated into a sequence of amino acids characteristic of a specific polypeptide.

An “isolated nucleic acid molecule” is a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

A “nucleic acid molecule construct” is a nucleic acid molecule, either single- or double-stranded, that has been modified through human intervention to contain segments of nucleic acid combined and juxtaposed in an arrangement not existing in nature.

“Linear DNA” denotes non-circular DNA molecules having free 5′ and 3′ ends. Linear DNA can be prepared from closed circular DNA molecules, such as plasmids, by enzymatic digestion or physical disruption.

“Complementary DNA (cDNA)” is a single-stranded DNA molecule that is formed from an mRNA template by the enzyme reverse transcriptase. Typically, a primer complementary to portions of MRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al, eds., Molecular Biology of the Gene, 4th ed. (The Benjamin/Cummings Publishing Company, Inc. 1987), and Lemaigre and Rousseau, Biochem. J. 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the TATA box and start of transcription. By this definition, a core promoter may or may not have detectable activity in the absence of specific sequences that may enhance the activity or confer tissue specific activity.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner.

An “enhancer” is a type of regulatory element that can increase the efficiency of transcription, regardless of the distance or orientation of the enhancer relative to the start site of transcription.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

A “polypeptide” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

A peptide or polypeptide encoded by a non-host DNA molecule is a “heterologous” peptide or polypeptide.

An “integrated genetic element” is a segment of DNA that has been incorporated into a chromosome of a host cell after that element is introduced into the cell through human manipulation. Within the present invention, integrated genetic elements are most commonly derived from linearized plasmids that are introduced into the cells by electroporation or other techniques. Integrated genetic elements are passed from the original host cell to its progeny.

A “cloning vector” is a nucleic acid molecule, such as a plasmid, cosmid, or bacteriophage, which has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector. In the present context, an example of a recombinant host is a cell that produces Zvn2R1 from an expression vector. In contrast, Zvn2R1 can be produced by a cell that is a “natural source” of Zvn2R1, and that lacks an expression vector.

“Integrative transformants” are recombinant host cells, in which heterologous DNA has become integrated into the genomic DNA of the cells.

A “fusion protein” is a hybrid protein expressed by a nucleic acid molecule comprising nucleotide sequences of at least two genes. For example, a fusion protein can comprise at least part of a Zvn2R1 polypeptide fused with a polypeptide that binds an affinity matrix. Such a fusion protein provides a means to isolate large quantities of Zvn2R1ligand using affinity chromatography.

The term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

The term “secretory signal sequence” denotes a DNA sequence that encodes a peptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “expression” refers to the biosynthesis of a gene product. For example, in the case of a structural gene, expression involves transcription of the structural gene into mRNA and the translation of MRNA into one or more polypeptides.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a polypeptide encoded by a splice variant of an mRNA transcribed from a gene.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of less than 10⁹ M⁻¹.

An “anti-idiotype antibody” is an antibody that binds with the variable region domain of an immunoglobulin. In the present context, an anti-idiotype antibody binds with the variable region of an anti-Zvn2R1 antibody, and thus, an anti-idiotype antibody mimics an epitope of Zvn2R1.

An “antibody fragment” is a portion of an antibody such as F(ab′)₂, F(ab)₂, Fab′, Fab, and the like. Regardless of structure, an antibody fragment binds with the same antigen that is recognized by the intact antibody. For example, an anti-Zvn2R1 monoclonal antibody fragment binds with an epitope of Zvn2R1.

The term “antibody fragment” also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

A “chimeric antibody” is a recombinant protein that contains the variable domains and complementary determining regions derived from a rodent antibody, while the remainder of the antibody molecule is derived from a human antibody.

“Humanized antibodies” are recombinant proteins in which murine complementarity determining regions of a monoclonal antibody have been transferred from heavy and light variable chains of the murine immunoglobulin into a human variable domain.

A “detectable label” is a molecule or atom, which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). DNA molecules encoding affinity tags are available from commercial suppliers (e.g., Pharmacia Biotech, Piscataway, N.J.).

A “naked antibody” is an entire antibody, as opposed to an antibody fragment, which is not conjugated with a therapeutic agent. Naked antibodies include both polyclonal and monoclonal antibodies, as well as certain recombinant antibodies, such as chimeric and humanized antibodies.

As used herein, the term “antibody component” includes both an entire antibody and an antibody fragment.

As used herein, the term “antibody fusion protein” refers to a recombinant molecule that comprises an antibody component and a Zvn2R1 polypeptide component. Examples of an antibody fusion protein include a protein that comprises a Zvn2R1 extracellular domain, and either an Fc domain or an antigen-biding region.

A “target polypeptide” or a “target peptide” is an amino acid sequence that comprises at least one epitope, and that is expressed on a target cell, such as a tumor cell, or a cell that carries an infectious agent antigen. T cells recognize peptide epitopes presented by a major histocompatibility complex molecule to a target polypeptide or target peptide and typically lyse the target cell or recruit other immune cells to the site of the target cell, thereby killing the target cell.

In eukaryotes, RNA polymerase II catalyzes the transcription of a structural gene to produce mRNA. A nucleic acid molecule can be designed to contain an RNA polymerase II template in which the RNA transcript has a sequence that is complementary to that of a specific mRNA. The RNA transcript is termed an “anti-sense RNA” and a nucleic acid molecule that encodes the anti-sense RNA is termed an “anti-sense gene.” Anti-sense RNA molecules are capable of binding to MRNA molecules, resulting in an inhibition of mRNA translation.

An “anti-sense oligonucleotide specific for Zvn2R1” or a “Zvn2R1 anti-sense oligonucleotide” is an oligonucleotide having a sequence (a) capable of forming a stable triplex with a portion of the Zvn2R1 gene, or (b) capable of forming a stable duplex with a portion of an mRNA transcript of the Zvn2R1 gene.

A “ribozyme” is a nucleic acid molecule that contains a catalytic center. The term includes RNA enzymes, self-splicing RNAs, self-cleaving RNAs, and nucleic acid molecules that perform these catalytic functions. A nucleic acid molecule that encodes a ribozyme is termed a “ribozyme gene.”

An “external guide sequence” is a nucleic acid molecule that directs the endogenous ribozyme, RNase P, to a particular species of intracellular mRNA, resulting in the cleavage of the mRNA by RNase P. A nucleic acid molecule that encodes an external guide sequence is termed an “external guide sequence gene.”

The term “variant Zvn2R1 gene” refers to nucleic acid molecules that encode a polypeptide comprising an amino acid sequence that is a modification of SEQ ID NOs:2 and 5. Such variants include naturally-occurring polymorphisms of Zvn2R1 genes, as well as synthetic genes that contain conservative amino acid substitutions of the amino acid sequence of SEQ ID NOs:2 and 5. Additional variant forms of Zvn2R1 genes are nucleic acid molecules that contain insertions or deletions of the nucleotide sequences described herein. A variant Zvn2R1 gene can be identified, for example, by determining whether the gene hybridizes with a nucleic acid molecule consisting of the nucleotide sequence of SEQ ID NOs:1 or 4, or their complements, under stringent conditions. The group of variant Zvn2R1 genes and polypeptides does not include the murine tissue-type vomeronasal putative pheromone receptor V2R2 mRNA (GenBank Accession No. AF053986).

Alternatively, variant Zvn2R1 genes can be identified by sequence comparison. Two amino acid sequences have “100% amino acid sequence identity” if the amino acid residues of the two amino acid sequences are the same when aligned for maximal correspondence. Similarly, two nucleotide sequences have “100% nucleotide sequence identity” if the nucleotide residues of the two nucleotide sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art (see, for example, Peruski and Peruski, The Internet and the New Biology:Tools for Genomic and Molecular Research (ASM Press, Inc. 1997), Wu et al. (eds.), “Information Superhighway and Computer Databases of Nucleic Acids and Proteins,” in Methods in Gene Biotechnology, pages 123-151 (CRC Press, Inc. 1997), and Bishop (ed.), Guide to Human Genome Computing, 2nd Edition (Academic Press, Inc. 1998)). Particular methods for determining sequence identity are described below.

Regardless of the particular method used to identify a variant Zvn2R1 gene or variant Zvn2R1 polypeptide, a variant gene or polypeptide encoded by a variant gene may be functionally characterized the ability to bind specifically to an anti-Zvn2R1 antibody.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

The present invention includes functional fragments of Zvn2R1 genes. Within the context of this invention, a “functional fragment” of a Zvn2R1 gene refers to a nucleic acid molecule that encodes a portion of a Zvn2R1 polypeptide, which is a domain described herein or at least specifically binds with an anti-Zvn2R1 antibody.

Due to the imprecision of standard analytical methods, molecular weights and lengths of polymers are understood to be approximate values. When such a value is expressed as “about” X or “approximately” X, the stated value of X will be understood to be accurate to ±10%.

3. Production of Zvn2R1 Coding Sequences

Nucleic acid molecules encoding a Zvn2R1 polypeptide can be obtained by screening a human cDNA or genomic library using polynucleotide probes based upon SEQ ID NOs:1 and 4. These techniques are standard and well-established.

As an illustration, a nucleic acid molecule that encodes a Zvn2R1 polypeptide can be isolated from a cDNA library. In this case, the first step would be to prepare the cDNA library by isolating RNA from a tissue, such as vomeronasal tissue, using methods well-known to those of skill in the art. In general, RNA isolation techniques must provide a method for breaking cells, a means of inhibiting RNase-directed degradation of RNA, and a method of separating RNA from DNA, protein, and polysaccharide contaminants. For example, total RNA can be isolated by freezing tissue in liquid nitrogen, grinding the frozen tissue with a mortar and pestle to lyse the cells, extracting the ground tissue with a solution of phenol/chloroform to remove proteins, and separating RNA from the remaining impurities by selective precipitation with lithium chloride (see, for example, Ausubel et al. (eds.), Short Protocols in Molecular Biology, 3^{rd} Edition, pages 4-1 to 4-6 (John Wiley & Sons 1995) [“Ausubel (1995)”]; Wu et al., Methods in Gene Biotechnology, pages 3341 (CRC Press, Inc. 1997) [“Wu (1997)”]).

Alternatively, total RNA can be isolated by extracting ground tissue with guanidinium isothiocyanate, extracting with organic solvents, and separating RNA from contaminants using differential centrifugation (see, for example, Chirgwin et al., Biochemistry 18:52 (1979); Ausubel (1995) at pages 4-1 to 4-6; Wu (1997) at pages 33-41).

In order to construct a cDNA library, poly(A)⁺ RNA must be isolated from a total RNA preparation. Poly(A)⁺ RNA can be isolated from total RNA using the standard technique of oligo(dT)-cellulose chromatography (see, for example, Aviv and Leder, Proc. Nat'l Acad. Sci. USA 69:1408 (1972); Ausubel (1995) at pages 4-11 to 4-12).

Double-stranded cDNA molecules are synthesized from poly(A)⁺ RNA using techniques well-known to those in the art. (see, for example, Wu (1997) at pages 41-46). Moreover, commercially available kits can be used to synthesize double-stranded cDNA molecules. For example, such kits are available from Life Technologies, Inc. (Gaithersburg, Md.), CLONTECH Laboratories, Inc. (Palo Alto, Calif.), Promega Corporation (Madison, Wis.) and STRATAGENE (La Jolla, Calif.).

Various cloning vectors are appropriate for the construction of a cDNA library. For example, a cDNA library can be prepared in a vector derived from bacteriophage, such as a λgt10 vector. See, for example, Huynh et al., “Constructing and Screening cDNA Libraries in λgt10 and λgt11,” in DNA Cloning:A Practical Approach Vol. 1, Glover (ed.), page 49 (IRL Press, 1985); Wu (1997) at pages 47-52.

Alternatively, double-stranded cDNA molecules can be inserted into a plasmid vector, such as a PBLUESCRIPT vector (STRATAGENE; La Jolla, Calif.), a LAMDAGEM-4 (Promega Corp.) or other commercially available vectors. Suitable cloning vectors also can be obtained from the American Type Culture Collection (Manassas, Va.).

To amplify the cloned cDNA molecules, the cDNA library is inserted into a prokaryotic host, using standard techniques. For example, a cDNA library can be introduced into competent E. coli DH5 cells, which can be obtained, for example, from Life Technologies, Inc. (Gaithersburg, Md.).

A human genomic library can be prepared by means well-known in the art (see, for example, Ausubel (1995) at pages 5-1 to 5-6; Wu (1997) at pages 307-327). Genomic DNA can be isolated by lysing tissue with the detergent Sarkosyl, digesting the lysate with proteinase K, clearing insoluble debris from the lysate by centrifugation, precipitating nucleic acid from the lysate using isopropanol, and purifying resuspended DNA on a cesium chloride density gradient.

DNA fragments that are suitable for the production of a genomic library can be obtained by the random shearing of genomic DNA or by the partial digestion of genomic DNA with restriction endonucleases. Genomic DNA fragments can be inserted into a vector, such as a bacteriophage or cosmid vector, in accordance with conventional techniques, such as the use of restriction enzyme digestion to provide appropriate termini, the use of alkaline phosphatase treatment to avoid undesirable joining of DNA molecules, and ligation with appropriate ligases. Techniques for such manipulation are well-known in the art (see, for example, Ausubel (1995) at pages 5-1 to 5-6; Wu (1997) at pages 307-327). Alternatively, human genomic libraries can be obtained from commercial sources such as Research Genetics (Huntsville, Ala.) and the American Type Culture Collection (Manassas, Va.).

A library containing cDNA or genomic clones can be screened with one or more polynucleotide probes based upon SEQ ID NO:1, using standard methods (see, for example, Ausubel (1995) at pages 6-1 to 6-11).

Nucleic acid molecules that encode a Zvn2R1 gene can also be obtained using the polymerase chain reaction (PCR) with oligonucleotide primers having nucleotide sequences that are based upon the nucleotide sequences of the Zvn2R1 gene, as described herein. General methods for screening libraries with PCR are provided by, for example, Yu et al., “Use of the Polymerase Chain Reaction to Screen Phage Libraries,” in Methods in Molecular Biology, Vol. 15:PCR Protocols:Current Methods and Applications, White (ed.), pages 211-215 (Humana Press, Inc. 1993). Moreover, techniques for using PCR to isolate related genes are described by, for example, Preston, “Use of Degenerate Oligonucleotide Primers and the Polymerase Chain Reaction to Clone Gene Family Members,” in Methods in Molecular Biology, Vol. 15: PCR Protocols:Current Methods and Applications, White (ed.), pages 317-337 (Humana Press, Inc. 1993).

Anti-Zvn2R1 antibodies, produced as described below, can also be used to isolate DNA sequences that encode Zvn2R1 polypeptides from cDNA libraries. For example, the antibodies can be used to screen λgt11 expression libraries, or the antibodies can be used for immunoscreening following hybrid selection and translation (see, for example, Ausubel (1995) at pages 6-12 to 6-16; Margolis et al., “Screening λ expression libraries with antibody and protein probes,” in DNA Cloning 2:Expression Systems, 2nd Edition, Glover et al. (eds.), pages 1-14 (Oxford University Press 1995)).

As an alternative, a Zvn2R1 gene can be obtained by synthesizing nucleic acid molecules using mutually priming long oligonucleotides and the nucleotide sequences described herein (see, for example, Ausubel (1995) at pages 8-8 to 8-9). Established techniques using the polymerase chain reaction provide the ability to synthesize DNA molecules at least two kilobases in length (Adang et al., Plant Molec. Biol. 21:1131 (1993), Bambot et al., PCR Methods and Applications 2:266 (1993), Dillon et al., “Use of the Polymerase Chain Reaction for the Rapid Construction of Synthetic Genes,” in Methods in Molecular Biology, Vol. 15: PCR Protocols:Current Methods and Applications, White (ed.), pages 263-268, (Humana Press, Inc. 1993), and Holowachuk et al., PCR Methods Appl. 4:299 (1995)).

The nucleic acid molecules of the present invention can also be synthesized with “gene machines” using protocols such as the phosphoramidite method. If chemically-synthesized double stranded DNA is required for an application such as the synthesis of a gene or a gene fragment, then each complementary strand is made separately. The production of short genes (60 to 80 base pairs) is technically straightforward and can be accomplished by synthesizing the complementary strands and then annealing them. For the production of longer genes (>300 base pairs), however, special strategies may be required, because the coupling efficiency of each cycle during chemical DNA synthesis is seldom 100%. To overcome this problem, synthetic genes (double-stranded) are assembled in modular form from single-stranded fragments that are from 20 to 100 nucleotides in length. For reviews on polynucleotide synthesis, see, for example, Glick and Pasternak, Molecular Biotechnology, Principles and Applications of Recombinant DNA (ASM Press 1994), Itakura et al., Annu. Rev. Biochem. 53:323 (1984), and Climie et al., Proc. Nat'l Acad. Sci. USA 87:633 (1990).

The sequence of a Zvn2R1 cDNA or Zvn2R1 genomic fragment can be determined using standard methods. Zvn2R1 polynucleotide sequences disclosed herein can also be used as probes or primers to clone 5′ non-coding regions of a Zvn2R1 gene. Promoter elements from a Zvn2R1 gene can be used to direct the expression of heterologous genes in, for example, transgenic animals or patients treated with gene therapy. The identification of genomic fragments containing a Zvn2R1 promoter or regulatory element can be achieved using well-established techniques, such as deletion analysis (see, generally, Ausubel (1995)).

Cloning of 5′ flanking sequences also facilitates production of Zvn2R1 proteins by “gene activation,” as disclosed in U.S. Pat. No. 5,641,670. Briefly, expression of an endogenous Zvn2R1 gene in a cell is altered by introducing into the Zvn2R1 locus a DNA construct comprising at least a targeting sequence, a regulatory sequence, an exon, and an unpaired splice donor site. The targeting sequence is a Zvn2R1 5′ non-coding sequence that permits homologous recombination of the construct with the endogenous Zvn2R1 locus, whereby the sequences within the construct become operably linked with the endogenous Zvn2R1 coding sequence. In this way, an endogenous Zvn2R1 promoter can be replaced or supplemented with other regulatory sequences to provide enhanced, tissue-specific, or otherwise regulated expression.

4. Production of Zvn2R1 Variants

The present invention provides a variety of nucleic acid molecules, including DNA and RNA molecules, which encode the Zvn2R1 polypeptides disclosed herein. Those skilled in the art will readily recognize that, in view of the degeneracy of the genetic code, considerable sequence variation is possible among these polynucleotide molecules. SEQ ID NO:3 is a degenerate nucleotide sequence that encompasses all nucleic acid molecules that encode the Zvn2R1 polypeptide of SEQ ID NO:2. Those skilled in the art will recognize that the degenerate sequence of SEQ ID NO:3 also provides all RNA sequences encoding SEQ ID NO:2, by substituting U for T. Similarly, the degenerate nucleotide sequences of SEQ ID NOs:6 and 9 encompass all nucleic acid molecules that encode the polypeptides of SEQ ID NOs:5 and 8, respectively. Thus, the present invention contemplates Zvn2R1 polypeptide-encoding nucleic acid molecules comprising the nucleotide sequence of SEQ ID NOs:1, 4, or 7, and RNA equivalents.

Table 2 sets forth the one-letter codes used within SEQ ID NOs:3, 6, and 9 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 2

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NOs:3, 6, and 9, encompassing all possible codons for a given amino acid, are set forth in Table 3.

TABLE 3

| | One | | |
|---|---|---|---|
| | One | | |
| | Letter | | Degenerate |
| Amino Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | F | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding an amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequences of SEQ ID NOs:2, 5, and 8. Variant sequences can be readily tested for functionality as described herein.

Different species can exhibit “preferential codon usage.” In general, see, Grantham et al, Nucl. Acids Res. 8:1893 (1980), Haas et al. Curr. Biol. 6:315 (1996), Wain-Hobson et al., Gene 13:355 (1981), Grosjean and Fiers, Gene 18:199 (1982), Holm, Nuc. Acids Res. 14:3075 (1986), Ikemura, J. Mol. Biol. 158:573 (1982), Sharp and Matassi, Curr. Opin. Genet Dev. 4:851 (1994), Kane, Curr. Opin. Biotechnol. 6:494 (1995), and Makrides, Microbiol. Rev. 60:512 (1996). As used herein, the term “preferential codon usage” or “preferential codons” is a term of art referring to protein translation codons that are most frequently used in cells of a certain species, thus favoring one or a few representatives of the possible codons encoding each amino acid (See Table 3). For example, the amino acid threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequences disclosed herein serve as a template for optimizing expression of polynucleotides in various cell types and species commonly used in the art and disclosed herein. Sequences containing preferential codons can be tested and optimized for expression in various species, and tested for functionality as disclosed herein.

The present invention further provides variant polypeptides and nucleic acid molecules that represent counterparts from other species (orthologs). These species include, but are not limited to mammalian, avian, amphibian, reptile, fish, insect and other vertebrate and invertebrate species. Of particular interest are Zvn2R1 polypeptides from other mammalian species, including rat, porcine, ovine, bovine, canine, feline, equine, and other primate polypeptides. Orthologs of human Zvn2R1 can be cloned using information and compositions provided by the present invention in combination with conventional cloning techniques. For example, a Zvn2R1 cDNA can be cloned using MRNA obtained from a tissue or cell type that expresses Zvn2R1 as disclosed herein. Suitable sources of mRNA can be identified by probing northern blots with probes designed from the sequences disclosed herein. A library is then prepared from mRNA of a positive tissue or cell line.

A Zvn2R1-encoding cDNA can be isolated by a variety of methods, such as by probing with a complete or partial cDNA or with one or more sets of degenerate probes based on the disclosed sequences. A cDNA can also be cloned using the polymerase chain reaction with primers designed from the representative human Zvn2R1 sequences disclosed herein. In addition, a cDNA library can be used to transform or transfect host cells, and expression of the cDNA of interest can be detected with an antibody to Zvn2R1 polypeptide.

Those skilled in the art will recognize that the sequence disclosed in SEQ ID NOs:1 and 4 represent a single allele of Zvn2R1, and that allelic variation and alternative splicing are expected to occur. Allelic variants of this sequence can be cloned by probing cDNA or genomic libraries from different individuals according to standard procedures. Allelic variants of the nucleotide sequences disclosed herein, including those containing silent mutations and those in which mutations result in amino acid sequence changes, are within the scope of the present invention, as are proteins, which are allelic variants of the amino acid sequences disclosed herein. cDNA molecules generated from alternatively spliced mRNAs, which retain the properties of the Zvn2R1 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

Within certain embodiments of the invention, the isolated nucleic acid molecules can hybridize under stringent conditions to nucleic acid molecules comprising nucleotide sequences disclosed herein. For example, such nucleic acid molecules can hybridize under stringent conditions to nucleic acid molecules comprising the nucleotide sequence of SEQ ID NOs:1 or 4, or to nucleic acid molecules comprising a nucleotide sequence complementary to SEQ ID NOs:1 or 4. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe.

A pair of nucleic acid molecules, such as DNA-DNA, RNA-RNA and DNA-RNA, can hybridize if the nucleotide sequences have some degree of complementarity. Hybrids can tolerate mismatched base pairs in the double helix, but the stability of the hybrid is influenced by the degree of mismatch. The Tₘ of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases. Stringent hybridization conditions encompass temperatures of about 5-25° C. below the Tₘ of the hybrid and a hybridization buffer having up to 1 M Na⁺. Higher degrees of stringency at lower temperatures can be achieved with the addition of formamide, which reduces the Tₘ of the hybrid about 1° C. for each 1% formamide in the buffer solution. Generally, such stringent conditions include temperatures of 20-70° C. and a hybridization buffer containing up to 6× SSC and 0-50% formamide. A higher degree of stringency can be achieved at temperatures of from 40-70° C. with a hybridization buffer having up to 4× SSC and from 0-50% formamide. Highly stringent conditions typically encompass temperatures of 42-70° C. with a hybridization buffer having up to 1× SSC and 0-50% formamide. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes.

The above conditions are meant to serve as a guide and it is well within the abilities of one skilled in the art to adapt these conditions for use with a particular polypeptide hybrid. The Tₘ for a specific target sequence is the temperature (under defined conditions) at which 50% of the target sequence will hybridize to a perfectly matched probe sequence. Those conditions that influence the Tₘ include, the size and base pair content of the polynucleotide probe, the ionic strength of the hybridization solution, and the presence of destabilizing agents in the hybridization solution. Numerous equations for calculating Tₘ are known in the art, and are specific for DNA, RNA and DNA-RNA hybrids and polynucleotide probe sequences of varying length (see, for example, Sambrook et al., Molecular Cloning:A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user-defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ. For smaller probes, <50 base pairs, hybridization is typically carried out at the Tₘ or 5- 10° C. below. This allows for the maximum rate of hybridization for DNA-DNA and DNA-RNA hybrids.

The length of the polynucleotide sequence influences the rate and stability of hybrid formation. Smaller probe sequences, <50 base pairs, reach equilibrium with complementary sequences rapidly, but may form less stable hybrids. Incubation times of anywhere from minutes to hours can be used to achieve hybrid formation. Longer probe sequences come to equilibrium more slowly, but form more stable complexes even at lower temperatures. Incubations are allowed to proceed overnight or longer. Generally, incubations are carried out for a period equal to three times the calculated Cot time. Cot time, the time it takes for the polynucleotide sequences to reassociate, can be calculated for a particular sequence by methods known in the art.

The base pair composition of polynucleotide sequence will effect the thermal stability of the hybrid complex, thereby influencing the choice of hybridization temperature and the ionic strength of the hybridization buffer. A-T pairs are less stable than G-C pairs in aqueous solutions containing sodium chloride. Therefore, the higher the G-C content, the more stable the hybrid. Even distribution of G and C residues within the sequence also contribute positively to hybrid stability. In addition, the base pair composition can be manipulated to alter the Tₘ of a given sequence. For example, 5-methyldeoxycytidine can be substituted for deoxycytidine and 5-bromodeoxuridine can be substituted for thymidine to increase the Tₘ, whereas 7-deazz-2′-deoxyguanosine can be substituted for guanosine to reduce dependence on Tₘ.

The ionic concentration of the hybridization buffer also affects the stability of the hybrid. Hybridization buffers generally contain blocking agents such as Denhardt's solution (Sigma Chemical Co., St. Louis, Mo.), denatured salmon sperm DNA, tRNA, milk powders (BLOTTO), heparin or SDS, and a Na⁺ source, such as SSC (1× SSC:0.15 M sodium chloride, 15 mM sodium citrate) or SSPE (1× SSPE:1.8 M NaCl, 10 mM NaH₂PO₄, 1 mM EDTA, pH 7.7). Typically, hybridization buffers contain from between 10 mM-1 M Na⁺. The addition of destabilizing or denaturing agents such as formamide, tetralkylammonium salts, guanidinium cations or thiocyanate cations to the hybridization solution will alter the Tₘ of a hybrid. Typically, formamide is used at a concentration of up to 50% to allow incubations to be carried out at more convenient and lower temperatures. Formamide also acts to reduce non-specific background when using RNA probes.

As an illustration, a nucleic acid molecule encoding a variant Zvn2R1 polypeptide can be hybridized with a nucleic acid molecule having the nucleotide sequence of SEQ ID NOs:1 or 4 (or their complements) at 42° C. overnight in a solution comprising 50% formamide, 5× SSC, 50 mM sodium phosphate (pH 7.6), 5× Denhardt's solution (100× Denhardt's solution: 2% (w/v) Ficoll 400, 2% (w/v) polyvinylpyrrolidone, and 2% (w/v) bovine serum albumin), 10% dextran sulfate, and 20 μg/ml denatured, sheared salmon sperm DNA. One of skill in the art can devise variations of these hybridization conditions. For example, the hybridization mixture can be incubated at a higher temperature, such as about 65° C., in a solution that does not contain formrnamide. Moreover, premixed hybridization solutions are available (e.g., EXPRESSHYB Hybridization Solution from CLONTECH Laboratories, Inc.), and hybridization can be performed according to the manufacturer's instructions.

Following hybridization, the nucleic acid molecules can be washed to remove non-hybridized nucleic acid molecules under stringent conditions, or under highly stringent conditions. Typical stringent washing conditions include washing in a solution of 0.5×- 2× SSC with 0.1% sodium dodecyl sulfate (SDS) at 55-65° C. As an illustration, nucleic acid molecules encoding a variant Zvn2R1 polypeptide remain hybridized with a nucleic acid molecule consisting of the nucleotide sequence of SEQ ID NOs:1 or 4 (or their complements) following stringent washing conditions, in which the wash stringency is equivalent to 0.5×- 2× SSC with 0.1% SDS at 55-65° C., including 0.5× SSC with 0.1% SDS at 55° C., or 2× SSC with 0.1% SDS at 65° C. One of skill in the art can readily devise equivalent conditions, for example, by substituting SSPE for SSC in the wash solution.

Typical highly stringent washing conditions include washing in a solution of 0.1×-0.2× SSC with 0.1% sodium dodecyl sulfate (SDS) at 50-65° C. For example, nucleic acid molecules encoding a variant Zvn2R1 polypeptide remain hybridized with a nucleic acid molecule consisting of the nucleotide sequence of NOs:1 or 4 (or their complements) following highly stringent washing conditions, in which the wash stringency is equivalent to 0.1×-0.2× SSC with 0.1% SDS at 50-65° C., including 0.1× SSC with 0.1% SDS at 50° C., or 0.2× SSC with 0.1% SDS at 65° C.

The present invention also provides isolated Zvn2R1 polypeptides that have a substantially similar sequence identity to the polypeptides of SEQ ID NOs:2 and 5, or their orthologs. The term “substantially similar sequence identity” is used herein to denote polypeptides having at least 70%, at least 80%, at least 90%, at least 95% or greater than 95% sequence identity to the sequences shown in SEQ ID NOs:2 and 5, or their orthologs.

The present invention also contemplates Zvn2R1 variant nucleic acid molecules that can be identified using two criteria:a determination of the similarity between the encoded polypeptide with the amino acid sequence of SEQ ID NOs:2 or 5, and a hybridization assay, as described above. Such Zvn2R1 variants include nucleic acid molecules (1) that remain hybridized with a nucleic acid molecule consisting of the nucleotide sequence of SEQ ID NOs:1 or 4 (or their complements) following stringent washing conditions, in which the wash stringency is equivalent to 0.5×-2× SSC with 0.1% SDS at 55-65° C., and (2) that encode a polypeptide having at least 70%, at least 80%, at least 90%, at least 95% or greater than 95% sequence identity to the amino acid sequence of SEQ ID NOs:2 or 5. Alternatively, Zvn2R1 variants can be characterized as nucleic acid molecules (1) that remain hybridized with a nucleic acid molecule consisting of the nucleotide sequence of SEQ ID NOs:1 or 4 (or their complements) following highly stringent washing conditions, in which the wash stringency is equivalent to 0.1×-0.2× SSC with 0.1% SDS at 50-65° C., and (2) that encode a polypeptide having at least 70%, at least 80%, at least 90%, at least 95% or greater than 95% sequence identity to the amino acid sequence of SEQ ID NOs:2 or 5.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 4 (amino acids are indicated by the standard one-letter codes). The percent identity is then calculated as: ([Total number of identical matches]/ [length of the longer sequence plus the number of gaps introduced into the longer sequence in order to align the two sequences])(100).

TABLE 4

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative Zvn2R1 variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as described above.

The present invention includes nucleic acid molecules that encode a polypeptide having a conservative amino acid change, compared with an amino acid sequence disclosed herein. For example, variants can be obtained that contain one or more amino acid substitutions of SEQ ID NOs:2 or 5, in which an alkyl amino acid is substituted for an alkyl amino acid in a Zvn2R1 amino acid sequence, an aromatic amino acid is substituted for an aromatic amino acid in a Zvn2R1 amino acid sequence, a sulfur-containing amino acid is substituted for a sulfur-containing amino acid in a Zvn2R1 amino acid sequence, a hydroxy-containing amino acid is substituted for a hydroxy-containing amino acid in a Zvn2R1 amino acid sequence, an acidic amino acid is substituted for an acidic amino acid in a Zvn2R1 amino acid sequence, a basic amino acid is substituted for a basic amino acid in a Zvn2R1 amino acid sequence, or a dibasic monocarboxylic amino acid is substituted for a dibasic monocarboxylic amino acid in a Zvn2R1 amino acid sequence. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Particular variants of Zvn2R1 are characterized by having at least 70%, at least 80%, at least 90%, at least 95% or greater than 95% sequence identity to the corresponding amino acid sequence (e.g., SEQ ID NOs:2 or 5), wherein the variation in amino acid sequence is due to one or more conservative amino acid substitutions.

Conservative amino acid changes in a Zvn2R1 gene can be introduced, for example, by substituting nucleotides for the nucleotides recited in SEQ ID NOs:1 or 4. Such “conservative amino acid” variants can be obtained by oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis:A Practical Approach (IRL Press 1991)). A variant Zvn2R1 polypeptide can be identified by the ability to specifically bind anti-Zvn2R1 antibodies.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is typically carried out in a cell-free system comprising an E. coli S30 extract and commercially available enzymes and other reagents. Proteins are purified by chromatography. See, for example, Robertson et al., J. Am. Chem. Soc. 113:2722 (1991), Ellman et al., Methods Enzymol. 202:301 (1991), Chung et al., Science 259:806 (1993), and Chung et al., Proc. Nat'l Acad. Sci. USA 90:10145 (1993).

In a second method, translation is carried out in Xenopus oocytes by microinjection of mutated mRNA and chemically aminoacylated suppressor tRNAs (Turcatti et al., J. Biol. Chem. 271:19991 (1996)). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470 (1994). Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395 (1993)).

A limited number of non-conservative amino acids, amino acids that are not encoded by the genetic code, non-naturally occurring amino acids, and unnatural amino acids may be substituted for Zvn2R1 amino acid residues.

Essential amino acids in the polypeptides of the present invention can be identified according to procedures known in the art, such as site-directed mutagenesis or alanine-scanning mutagenesis (Cunningham and Wells, Science 244:1081 (1989), Bass et al., Proc. Nat'l Acad. Sci. USA 88:4498 (1991), Coombs and Corey, “Site-Directed Mutagenesis and Protein Engineering,” in Proteins:Analysis and Design, Angeletti (ed.), pages 259-311 (Academic Press, Inc. 1998)). In the latter technique, single alanine mutations are introduced at every residue in the molecule, and the resultant mutant molecules are tested for biological activity to identify amino acid residues that are critical to the activity of the molecule. See also, Hilton et al., J. Biol. Chem. 271:4699 (1996).

Although sequence analysis can be used to further define the Zvn2R1 ligand binding region, amino acids that play a role in Zvn2R1 binding activity can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306 (1992), Smith et al., J. Mol. Biol. 224:899 (1992), and Wlodaver et al., FEBS Lett. 309:59 (1992).

Multiple amino acid substitutions can be made and tested using known methods of mutagenesis and screening, such as those disclosed by Reidhaar-Olson and Sauer (Science 241:53 (1988)) or Bowie and Sauer (Proc. Nat'l Acad. Sci. USA 86:2152 (1989)). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832 (1991), Ladner et al., U.S. Pat. No. 5,223,409, Huse, international publication No. WO 92/06204, and region-directed mutagenesis (Derbyshire et al., Gene 46:145 (1986), and Ner et al., DNA 7:127, (1988)). Moreover, Zvn2R1 labeled with biotin or FITC can be used for expression cloning.

Variants of the disclosed Zvn2R1 nucleotide and polypeptide sequences can also be generated through DNA shuffling as disclosed by Stemmer, Nature 370:389 (1994), Stemmer, Proc. Nat'l Acad. Sci. USA 91:10747 (1994), and international publication No. WO 97/20078. Briefly, variant DNA molecules are generated by in vitro homologous recombination by random fragmentation of a parent DNA followed by reassembly using PCR, resulting in randomly introduced point mutations. This technique can be modified by using a family of parent DNA molecules, such as allelic variants or DNA molecules from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

While one general class of Zvn2R1 variants are polypeptides having an amino acid sequence that is a mutation of the amino acid sequences disclosed herein, another general class of Zvn2R1 variants is provided by anti-idiotype antibodies, and fragments thereof, as described below. Moreover, recombinant antibodies comprising anti-idiotype variable domains can be used as variants (see, for example, Monfardini et al., Proc. Assoc. Am. Physicians 108:420 (1996)). Since the variable domains of anti-idiotype Zvn2R1 antibodies mimic Zvn2R1, these domains can provide Zvn2R1 binding activity. Methods of producing anti-idiotypic catalytic antibodies are known to those of skill in the art (see, for example, Joron et al., Ann. N Y Acad. Sci. 672:216 (1992), Friboulet et al., Appl. Biochem. Biotechnol. 47:229 (1994), and Avalle et al., Ann. N Y Acad. Sci. 864:118 (1998)).

Another approach to identifying Zvn2R1 variants is provided by the use of combinatorial libraries. Methods for constructing and screening phage display and other combinatorial libraries are provided, for example, by Kay et al., Phage Display of Peptides and Proteins (Academic Press 1996), Verdine, U.S. Pat. No. 5,783,384, Kay, et. al., U.S. Pat. No. 5,747,334, and Kauffman et al., U.S. Pat. No. 5,723,323.

Mutagenesis methods as disclosed herein can be combined with high-throughput, automated screening methods to detect activity of cloned, mutagenized polypeptides in host cells. Mutagenized DNA molecules that encode biologically active polypeptides, or polypeptides that bind with anti-Zvn2R1 antibodies, can be recovered from the host cells and rapidly sequenced using modern equipment. These methods allow the rapid determination of the importance of individual amino acid residues in a polypeptide of interest, and can be applied to polypeptides of unknown structure.

The present invention also includes “functional fragments” of Zvn2R1 polypeptides and nucleic acid molecules encoding such functional fragments. Routine deletion analyses of nucleic acid molecules can be performed to obtain functional fragments of a nucleic acid molecule that encodes a Zvn2R1 polypeptide. As an illustration, DNA molecules having the nucleotide sequence of SEQ ID NO:1 can be digested with Bal31 nuclease to obtain a series of nested deletions. The fragments are then inserted into expression vectors in proper reading frame, and the expressed polypeptides are isolated and tested for the ability to bind anti-Zvn2R1 antibodies. One alternative to exonuclease digestion is to use oligonucleotide-directed mutagenesis to introduce deletions or stop codons to specify production of a desired fragment. Alternatively, particular fragments of a Zvn2R1 gene can be synthesized using the polymerase chain reaction.

As an illustration of this general approach, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993), Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987), Herschman, “The EGF Receptor,” in Control of Animal Cell Proliferation, Vol. 1, Boynton et al., (eds.) pages 169-199 (Academic Press 1985), Coumailleau et al., J. Biol. Chem. 270:29270 (1995); Fukunaga et al., J. Biol. Chem. 270:25291 (1995); Yamaguchi et al., Biochem. Pharmacol. 50:1295 (1995), and Meisel et al., Plant Molec. Biol. 30:1 (1996).

The present invention also contemplates functional fragments of a Zvn2R1 gene that have amino acid changes, compared with an amino acid sequence disclosed herein. A variant Zvn2R1 gene can be identified on the basis of structure by determining the level of identity with disclosed nucleotide and amino acid sequences, as discussed above. An alternative approach to identifying a variant gene on the basis of structure is to determine whether a nucleic acid molecule encoding a potential variant Zvn2R1 gene can hybridize to a nucleic acid molecule comprising a nucleotide sequence, such as SEQ ID NOs:1 or 4.

The present invention also provides polypeptide fragments or peptides comprising an epitope-bearing portion of a Zvn2R1 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983)).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660 (1983)). Accordingly, antigenic epitope-bearing peptides and polypeptides of the present invention are useful to raise antibodies that bind with the polypeptides described herein.

Antigenic epitope-bearing peptides and polypeptides can contain at least four to ten amino acids, at least ten to fifteen amino acids, or about 15 to about 30 amino acids of an amino acid sequence disclosed herein. Such epitope-bearing peptides and polypeptides can be produced by fragmenting a Zvn2R1 polypeptide, or by chemical peptide synthesis, as described herein. Moreover, epitopes can be selected by phage display of random peptide libraries (see, for example, Lane and Stephen, Curr.

Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies:Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

For any Zvn2R1 polypeptide, including variants and fusion proteins, one of ordinary skill in the art can readily generate a fully degenerate polynucleotide sequence encoding that variant using the information set forth in Tables 2 and 3 above. Moreover, those of skill in the art can use standard software to devise Zvn2R1 variants based upon the nucleotide and amino acid sequences described herein. Accordingly, the present invention includes a computer-readable medium encoded with a data structure that provides at least one of the following sequences: SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9. Suitable forms of computer-readable media include magnetic media and optically-readable media. Examples of magnetic media include a hard or fixed drive, a random access memory (RAM) chip, a floppy disk, digital linear tape (DLT), a disk cache, and a ZIP disk. Optically readable media are exemplified by compact discs (e.g., CD-read only memory (ROM), CD-rewritable (RW), and CD-recordable), and digital versatile/video discs (DVD) (e.g., DVD-ROM, DVD-RAM, and DVD+RW).

5. Production of Zvn2R1 Polypeptides

The polypeptides of the present invention, including full-length polypeptides, functional fragments, and fusion proteins, can be produced in recombinant host cells following conventional techniques. To express a Zvn2R1 gene, a nucleic acid molecule encoding the polypeptide must be operably linked to regulatory sequences that control transcriptional expression in an expression vector and then, introduced into a host cell. In addition to transcriptional regulatory sequences, such as promoters and enhancers, expression vectors can include translational regulatory sequences and a marker gene, which is suitable for selection of cells that carry the expression vector.

Expression vectors that are suitable for production of a foreign protein in eukaryotic cells typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence. As discussed above, expression vectors can also include nucleotide sequences encoding a secretory sequence that directs the heterologous polypeptide into the secretory pathway of a host cell. For example, a Zvn2R1 expression vector may comprise a Zvn2R1 gene and a secretory sequence derived from any secreted gene.

Zvn2R1 proteins of the present invention may be produced in mammalian cells. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 (Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658).

For a mammalian host, the transcriptional and translational regulatory signals may be derived from viral sources, such as adenovirus, bovine papilloma virus, simian virus, or the like, in which the regulatory signals are associated with a particular gene, which has a high level of expression. Suitable transcriptional and translational regulatory sequences also can be obtained from mammalian genes, such as actin, collagen, myosin, and metallothionein genes.

Transcriptional regulatory sequences include a promoter region sufficient to direct the initiation of RNA synthesis. Suitable eukaryotic promoters include the promoter of the mouse metallothionein I gene (Hamer et al., J. Molec. Appl. Genet. 1:273 (1982)), the TK promoter of Herpes virus (McKnight, Cell 31:355 (1982)), the SV40 early promoter (Benoist et al., Nature 290:304 (1981)), the Rous sarcoma virus promoter (Gorman et al., Proc. Nat'l Acad. Sci. USA 79:6777 (1982)), the cytomegalovirus promoter (Foecking et al., Gene 45:101 (1980)), and the mouse mammary tumor virus promoter (see, generally, Etcheverry, “Expression of Engineered Proteins in Mammalian Cell Culture,” in Protein Engineering:Principles and Practice, Cleland et al. (eds.), pages 163-181 (John Wiley & Sons, Inc. 1996)).

Alternatively, a prokaryotic promoter, such as the bacteriophage T3 RNA polymerase promoter, can be used to control Zvn2R1 gene expression in mammalian cells if the prokaryotic promoter is regulated by a eukaryotic promoter (Zhou et al., Mol. Cell. Biol. 10:4529 (1990), and Kaufman et al., Nucl. Acids Res. 19:4485 (1991)).

An expression vector can be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

For example, one suitable selectable marker is a gene that provides resistance to the antibiotic neomycin. In this case, selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A suitable amplifiable selectable marker is dihydrofolate reductase, which confers resistance to methotrexate. Other drug resistance genes (e.g., hygromycin resistance, multi-drug resistance, puromycin acetyltransferase) can also be used. Alternatively, markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Zvn2R1 polypeptides can also be produced by cultured mammalian cells using a viral delivery system. Exemplary viruses for this purpose include adenovirus, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161 (1994), and Douglas and Curiel, Science & Medicine 4:44 (1997)). Advantages of the adenovirus system include the accommodation of relatively large DNA inserts, the ability to grow to high-titer, the ability to infect a broad range of mammalian cell types, and flexibility that allows use with a large number of available vectors containing different promoters.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. An option is to delete the essential E1 gene from the viral vector, which results in the inability to replicate unless the E1 gene is provided by the host cell. Adenovirus vector-infected human 293 cells (ATCC Nos. CRL-1573, 45504, 45505), for example, can be grown as adherent cells or in suspension culture at relatively high cell density to produce significant amounts of protein (see Garnier et al., Cytotechnol. 15:145 (1994)).

Zvn2R1 can also be produced in other higher eukaryotic cells, such as avian, fungal, insect, yeast, or plant cells. The baculovirus system provides an efficient means to introduce cloned Zvn2R1 genes into insect cells. Suitable expression vectors are based upon the Autographa californica multiple nuclear polyhedrosis virus (AcMNPV), and contain well-known promoters such as Drosophila heat shock protein (hsp) 70 promoter, Autographa californica nuclear polyhedrosis virus immediate-early gene promoter (IE-1) and the delayed early 39K promoter, baculovirus p10 promoter, and the Drosophila metallothionein promoter. A second method of making recombinant baculovirus utilizes a transposon-based system described by Luckow (Luckow, et al., J. Virol. 67:4566 (1993)). This system, which utilizes transfer vectors, is sold in the BAC-to-BAC kit (Life Technologies, Rockville, Md.). This system utilizes a transfer vector, PFASTBAC (Life Technologies) containing a Tn7 transposon to move the DNA encoding the Zvn2R1 polypeptide into a baculovirus genome maintained in E. coli as a large plasmid called a “bacmid.” See, Hill-Perkins and Possee, J. Gen. Virol. 71:971 (1990), Bonning, et al., J. Gen. Virol. 75:1551 (1994), and Chazenbalk, and Rapoport, J. Biol. Chem. 270:1543 (1995). In addition, transfer vectors can include an in-frame fusion with DNA encoding an epitope tag at the C- or N-terminus of the Zvn2R1 polypeptide, for example, a Glu-Glu epitope tag (Grussenmeyer et al., Proc. Nat'l Acad. Sci. 82:7952 (1985)). Using a technique known in the art, a transfer vector containing a Zvn2R1 gene is transformed into E. coli, and screened for bacmids, which contain an interrupted lacZ gene indicative of recombinant baculovirus. The bacmid DNA containing the recombinant baculovirus genome is then isolated using common techniques.

The illustrative PFASTBAC vector can be modified to a considerable degree. For example, the polyhedrin promoter can be removed and substituted with the baculovirus basic protein promoter (also known as Pcor, p6.9 or MP promoter), which is expressed earlier in the baculovirus infection, and has been shown to be advantageous for expressing secreted proteins (see, for example, Hill-Perkins and Possee, J. Gen. Virol. 71:971 (1990), Bonning, et al., J. Gen. Virol. 75:1551 (1994), and Chazenbalk and Rapoport, J. Biol. Chem. 270:1543 (1995). In such transfer vector constructs, a short or long version of the basic protein promoter can be used. Moreover, transfer vectors can be constructed, which replace the native Zvn2R1 secretory signal sequences with secretory signal sequences derived from insect proteins. For example, a secretory signal sequence from Ecdysteroid Glucosyltransferase (EGT), honey bee Melittin (Invitrogen Corporation; Carlsbad, Calif.), or baculovirus gp67 (PharMingen: San Diego, Calif.) can be used in constructs to replace the native Zvn2Rl secretory signal sequence.

The recombinant virus or bacmid is used to transfect host cells. Suitable insect host cells include cell lines derived from IPLB-Sf-21, a Spodoptera frugiperda pupal ovarian cell line, such as Sf9 (ATCC CRL 1711), Sf21 AE, and Sf21 (Invitrogen Corporation; San Diego, Calif.), as well as Drosophila Schneider-2 cells, and the HIGH FIVEO cell line (Invitrogen) derived from Trichoplusia ni (U.S. Pat. No. 5,300,435). Commercially available serum-free media can be used to grow and to maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. When recombinant virus is used, the cells are typically grown up from an inoculation density of approximately 2-5 ×10⁵ cells to a density of 1-2×10⁶ cells at which time a recombinant viral stock is added at a multiplicity of infection (MOI) of 0.1 to 10, more typically near 3.

Established techniques for producing recombinant proteins in baculovirus systems are provided by Bailey et al., “Manipulation of Baculovirus Vectors,” in Methods in Molecular Biology, Volume 7:Gene Transfer and Expression Protocols, Murray (ed.), pages 147-168 (The Humana Press, Inc. 1991), by Patel et al., “The baculovirus expression system,” in DNA Cloning 2:Expression Systems, 2nd Edition, Glover et al. (eds.), pages 205-244 (Oxford University Press 1995), by Ausubel (1995) at pages 16-37 to 16-57, by Richardson (ed.), Baculovirus Expression Protocols (The Humana Press, Inc. 1995), and by Lucknow, “Insect Cell Expression Technology,” in Protein Engineering:Principles and Practice, Cleland et al. (eds.), pages 183-218 (John Wiley & Sons, Inc. 1996).

Fungal cells, including yeast cells, can also be used to express the genes described herein. Yeast species of particular interest in this regard include Saccharomyces cerevisiae, Pichia pastoris, and Pichia methanolica. Suitable promoters for expression in yeast include promoters from GAL1 (galactose), PGK (phosphoglycerate kinase), ADH (alcohol dehydrogenase), AOX1 (alcohol oxidase), HIS4 (histidinol dehydrogenase), and the like. Many yeast cloning vectors have been designed and are readily available. These vectors include YIp-based vectors, such as YIp5, YRp vectors, such as YRp17, YEp vectors such as YEp13 and YCp vectors, such as YCp19. Methods for transforming S. cerevisiae cells with exogenous DNA and producing recombinant polypeptides therefrom are disclosed by, for example, Kawasaki, U.S. Pat. No. 4,599,311, Kawasaki et al., U.S. Pat. No. 4,931,373, Brake, U.S. Pat. No. 4,870,008, Welch et al., U.S. Pat. No. 5,037,743, and Murray et al., U.S. Pat. No. 4,845,075. Transformed cells are selected by phenotype determined by the selectable marker, commonly drug resistance or the ability to grow in the absence of a particular nutrient (e.g., leucine). A suitable vector system for use in Saccharomyces cerevisiae is the POT1 vector system disclosed by Kawasaki et al. (U.S. Pat. No. 4,931,373), which allows transformed cells to be selected by growth in glucose-containing media. Additional suitable promoters and terminators for use in yeast include those from glycolytic enzyme genes (see, e.g., Kawasaki, U.S. Pat. No. 4,599,311, Kingsman et al., U.S. Pat. No. 4,615,974, and Bitter, U.S. Pat. No. 4,977,092) and alcohol dehydrogenase genes. See also U.S. Pat. Nos. 4,990,446, 5,063,154, 5,139,936, and 4,661,454.

Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459 (1986), and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

For example, the use of Pichia methanolica as host for the production of recombinant proteins is disclosed by Raymond, U.S. Pat. No. 5,716,808, Raymond, U.S. Pat. No. 5,736,383, Raymond et al., Yeast 14:11-23 (1998), and in international publication Nos. WO 97/17450, WO 97/17451, WO 98/02536, and WO 98/02565. DNA molecules for use in transforming P. methanolica will commonly be prepared as double-stranded, circular plasmids, which can be linearized prior to transformation. For polypeptide production in P. methanolica, the promoter and terminator in the plasmid can be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, the entire expression segment of the plasmid can be flanked at both ends by host DNA sequences. A suitable selectable marker for use in Pichia methanolica is a P. methanolica ADE2 gene, which encodes phosphoribosyl-5-aminoimidazole carboxylase (AIRC; EC 4.1.1.21), and which allows ade2 host cells to grow in the absence of adenine. For large-scale, industrial processes where it is desirable to minimize the use of methanol, host cells can be used in which both methanol utilization genes (AUG1 and AUG2) are deleted. For production of secreted proteins, host cells can be deficient in vacuolar protease genes (PEP4 and PRB1). Electroporation is used to facilitate the introduction of a plasmid containing DNA encoding a polypeptide of interest into P. methanolica cells. P. methanolica cells can be transformed by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Expression vectors can also be introduced into plant protoplasts, intact plant tissues, or isolated plant cells. Methods for introducing expression vectors into plant tissue include the direct infection or co-cultivation of plant tissue with Agrobacterium tumefaciens, microprojectile-mediated delivery, DNA injection, electroporation, and the like. See, for example, Horsch et al., Science 227:1229 (1985), Klein et al., Biotechnology 10:268 (1992), and Miki et al., “Procedures for Introducing Foreign DNA into Plants,” in Methods in Plant Molecular Biology and Biotechnology, Glick et al. (eds.), pages 67-88 (CRC Press, 1993).

Alternatively, Zvn2R1 genes can be expressed in prokaryotic host cells. Suitable promoters that can be used to produce Zvn2R1 polypeptides in a prokaryotic host are well-known to those of skill in the art and include promoters capable of recognizing the T4, T3, Sp6 and T7 polymerases, the PR and PL promoters of bacteriophage lambda, the trp, recA, heat shock, lacUV5, tac, lpp-lacSpr, phoA, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. Prokaryotic promoters have been reviewed by Glick, J. Ind. Microbiol. 1:277 (1987), Watson et al., Molecular Biology of the Gene, 4th Ed. (Benjamin Cummins 1987), and by Ausubel et al. (1995).

Illustrative prokaryotic hosts include E. coli and Bacillus subtilus. Suitable strains of E. coli include BL21(DE3), BL21(DE3)pLysS, BL21(DE3)pLysE, DH1, DH4I, DH5, DH5I, DH5IF′, DH5IMCR, DH10B, DH10B/p3, DH11S, C600, HB101, JM101, JM105, JM109, JM110, K38, RR1, Y1088, Y1089, CSH18, ER1451, and ER1647 (see, for example, Brown (ed.), Molecular Biology Labfax (Academic Press 1991)). Suitable strains of Bacillus subtilus include BR151, YB886, MI119, MI120, and B170 (see, for example, Hardy, “Bacillus Cloning Methods,” in DNA Cloning:A Practical Approach, Glover (ed.) (IRL Press 1985)).

When producing a Zvn2R1 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Methods for producing proteins in prokaryotic hosts are well-known to those of skill in the art (see, for example, Williams et aL, “Expression of foreign proteins in E. coli using plasmid vectors and purification of specific polyclonal antibodies,” in DNA Cloning 2:Expression Systems, 2nd Edition, Glover et al. (eds.), page 15 (Oxford University Press 1995), Ward et al., “Genetic Manipulation and Expression of Antibodies,” in Monoclonal Antibodies:Principles and Applications, page 137 (Wiley-Liss, Inc. 1995), and Georgiou, “Expression of Proteins in Bacteria,” in Protein Engineering:Principles and Practice, Cleland et al. (eds.), page 101 (John Wiley & Sons, Inc. 1996)).

Standard methods for introducing expression vectors into bacterial, yeast, insect, and plant cells are provided, for example, by Ausubel (1995).

General methods for producing and recovering foreign protein produced by a mammalian cell system are provided by, for example, Etcheverry, “Expression of Engineered Proteins in Mammalian Cell Culture,” in Protein Engineering:Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2:Expression Systems, 2nd Edition, Glover et al. (eds.), pages 59-92 (Oxford University Press 1995). Established methods for isolating recombinant proteins from a baculovirus system are described by Richardson (ed.), Baculovirus Expression Protocols (The Humana Press, Inc. 1995).

As an alternative, polypeptides of the present invention can be synthesized by exclusive solid phase synthesis, partial solid phase methods, fragment condensation or classical solution synthesis. These synthesis methods are well-known to those of skill in the art (see, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept Prot. 3:3 (1986), Atherton et aL., Solid Phase Peptide Synthesis:A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, for example, Dawson et al., Science 266:776 (1994), Hackeng et al., Proc. Nat'l Acad. Sci. USA 94:7845 (1997), Dawson, Methods Enzymol. 287: 34 (1997), Muir et al, Proc. Nat'l Acad. Sci. USA 95:6705 (1998), and Severinov and Muir, J. Biol. Chem. 273:16205 (1998)).

General methods for isolating seven transmembrane domain receptors are known to those of skill in the art. See, for example, Strosberg and Fernandes, “Recombinant G-Protein-coupled receptors as screening tools for drug discovery,” in Leff, P. (Ed.), Receptor-Based Drug Design (Marcel Dekker, Inc. 1998), Kilpatrick et al., Trends Phann. Sci. 20:294 (1999), and Lynch, K. R. (Ed.), Identification and Expression of G Protein-Coupled Receptors (Wiley-Liss, 1999).

Peptides and polypeptides of the present invention comprise at least six, at least nine, or at least 15 contiguous amino acid residues of SEQ ID NOs:2 or 5, or a segment of the amino acid sequence of SEQ ID NOs:2 or 5. Such segments include amino acid residues of SEQ ID NO:5 assigned to a domain, as shown in Table 1. Within certain embodiments of the invention, the polypeptides comprise 20, 30, 40, 50, 100, or more contiguous residues of these amino acid sequences. Nucleic acid molecules encoding such peptides and polypeptides are useful as polymerase chain reaction primers and probes.

6. Production of Zvn2R1 Fusion Proteins and Conjugates

Fusion proteins of Zvn2R1 can be used to produce Zvn2R1 in a recombinant host, and to isolate the produced Zvn2R1. One type of fusion protein comprises a peptide that guides a Zvn2R1 polypeptide from a recombinant host cell. To direct a Zvn2R1 polypeptide into the secretory pathway of a eukaryotic host cell, a secretory signal sequence (also known as a signal peptide, a leader sequence, prepro sequence or pre sequence) is provided in the Zvn2R1 expression vector. While the secretory signal sequence may be derived from Zvn2R1, a suitable signal sequence may also be derived from another secreted protein or synthesized de novo. The secretory signal sequence is operably linked to a Zvn2R1-encoding sequence such that the two sequences are joined in the correct reading frame and positioned to direct the newly synthesized polypeptide into the secretory pathway of the host cell. Secretory signal sequences are commonly positioned 5′ to the nucleotide sequence encoding the polypeptide of interest, although certain secretory signal sequences may be positioned elsewhere in the nucleotide sequence of interest (see, e.g., Welch et al., U.S. Pat. No. 5,037,743; Holland et al., U.S. Pat. No. 5,143,830).

Although the secretory signal sequence of Zvn2R 1 or another protein produced by mammalian cells (e.g., tissue-type plasminogen activator signal sequence, as described, for example, in U.S. Pat. No. 5,641,655) is useful for production of Zvn2R1 in recombinant mammalian hosts, a yeast signal sequence is preferred for expression in yeast cells. Examples of suitable yeast signal sequences are those derived from yeast mating phermone factor (encoded by the MFal gene), invertase (encoded by the SUC2 gene), or acid phosphatase (encoded by the PHO5 gene). See, for example, Romanos et al., “Expression of Cloned Genes in Yeast,” in DNA Cloning 2:A Practical Approach, 2nd Edition, Glover and Hames (eds.), pages 123-167 (Oxford University Press 1995).

Peptide tags that are useful for isolating heterologous polypeptides produced by either prokaryotic or eukaryotic cells include polyHistidine tags (which have an affinity for nickel-chelating resin), c-myc tags, calmodulin binding protein (isolated with calmodulin affinity chromatography), substance P, the RYIRS tag (which binds with anti-RYIRS antibodies), the Glu-Glu tag, and the FLAG tag (which binds with anti-FLAG antibodies). See, for example, Luo et al., Arch. Biochem. Biophys. 329:215 (1996), Morganti et al., Biotechnol. Appl. Biochem. 23:67 (1996), and Zheng et al., Gene 186:55 (1997). Nucleic acid molecules encoding such peptide tags are available, for example, from Sigma-Aldrich Corporation (St. Louis, Mo.).

The present invention further provides a variety of other polypeptide fusions. For example, part or all of a domain(s) conferring a biological function can be swapped between Zvn2R1 of the present invention with the functionally equivalent domain(s) from another member of the vomeronasal receptor family. Polypeptide fusions can be produced in recombinant host cells to produce a variety of Zvn2R1 fusion analogs. A Zvn2R1 polypeptide can be fused to two or more moieties or domains, such as an affinity tag for purification and a targeting domain. Polypeptide fusions can also comprise one or more cleavage sites, particularly between domains. See, for example, Tuan et al., Connective Tissue Research 34:1 (1996).

Another form of fusion protein comprises a Zvn2R1 polypeptide and an immunoglobulin heavy chain constant region, typically an Fc fragment, which contains two or three constant region domains and a hinge region but lacks the variable region. As an illustration, Chang et al., U.S. Pat. No. 5,723,125, describe a fusion protein comprising a human interferon and a human immunoglobulin Fe fragment. The C-terminal of the interferon is linked to the N-terminal of the Fc fragment by a peptide linker moiety. An example of a peptide linker is a peptide comprising primarily a T cell inert sequence, which is immunologically inert. An exemplary peptide linker has the amino acid sequence: GGSGG SGGGG SGGGG S (SEQ ID NO:10). In this fusion protein, an illustrative Fe moiety is a human λ4 chain, which is stable in solution and has little or no complement activating activity. Accordingly, the present invention contemplates a Zvn2R1 fusion protein that comprises a Zvn2R1 moiety and a human Fe fragment, wherein the C-terminus of the Zvn2R1 moiety is attached to the N-terminus of the Fe fragment via a peptide linker, such as a peptide consisting of the amino acid sequence of SEQ ID NO:10. The Zvn2R1 moiety can be a Zvn2R1 molecule or a fragment thereof. For example, a fusion protein can comprise a ligand binding domain of Zvn2R 1, such as an amino acid sequence comprising amino acid residues 30 to about 610 of SEQ ID NO:8, and an Fe fragment (e.g., a human Fe fragment).

A dimeric Zvn2R1 Fe protein may exhibit increased binding avidity for a Zvn2R1 ligand due to the divalent juxapostion of two Zvn2R1 ligand-binding domains effected by the Fe protein scaffold. Dimerization of the calcium and metabotropic glutamate receptor ligand-binding domains have been shown to be important for ligand-binding (Okamoto et al., J. Biol. Chem. 272:13089 (1998); Han and Hampson, J. Biol. Chem. 274:10008 (1999); Goldsmith et al., J. Biol. Cliem. 274:11303 (1999)).

In another variation, a Zvn2R1 fusion protein comprises an IgG sequence, a Zvn2R1 moiety covalently joined to the aminoterminal end of the IgG sequence, and a signal peptide that is covalently joined to the aminoterminal of the Zvn2R1 moiety, wherein the IgG sequence consists of the following elements in the following order: a hinge region, a CH₂ domain, and a CH₃ domain. Accordingly, the IgG sequence lacks a CH₁ domain. The Zvn2R1 moiety displays a Zvn2R1 activity, as described herein, such as the ability to bind with a Zvn2R1 ligand. This general approach to producing fusion proteins that comprise both antibody and nonantibody portions has been described by LaRochelle et al., EP 742830 (WO 95/21258).

Other examples of antibody fusion proteins include polypeptides that comprise an antigen-binding domain and a Zvn2R1 fragment that contains a Zvn2R1 extracellular domain. Such molecules can be used to target particular tissues for the benefit of Zvn2R1 binding activity.

Fusion proteins comprising a Zvn2R1 moiety and an Fe moiety can be used, for example, as an in vitro assay tool. For example, the presence of a Zvn2R1 ligand in a biological sample can be detected using a Zvn2R1-immunoglobulin fusion protein, in which the Zvn2R1 moiety is used to bind the ligand, and a macromolecule, such as Protein A or anti-Fc antibody, is used to bind the fusion protein to a solid support. Such systems can be used to identify a Zvn2R1 ligand or molecules that interfere with the binding of a Zvn2R1 ligand to its receptor.

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. General methods for enzymatic and chemical cleavage of fusion proteins are described, for example, by Ausubel (1995) at pages 16-19 to 16-25.

Zvn2R1 polypeptides may be monomers or multimers; glycosylated or non-glycosylated; PEGylated or non-PEGylated; and may or may not include an initial methionine amino acid residue.

The present invention also contemplates chemically modified Zvn2R1 compositions, in which a Zvn2R1 polypeptide is linked with a polymer. Illustrative Zvn2R1 polypeptides include soluble polypeptides that lack a functional transmembrane domain, such as a polypeptide comprising amino acid residues 30 to about 610 of SEQ ID NO:8. Typically, the polymer is water-soluble so that the Zvn2R1 conjugate does not precipitate in an aqueous environment, such as a physiological environment. An example of a suitable polymer is one that has been modified to have a single reactive group, such as an active ester for acylation, or an aldehyde for alkylation, In this way, the degree of polymerization can be controlled. An example of a reactive aldehyde is polyethylene glycol propionaldehyde, or mono-(C₁-C₁₀) alkoxy, or aryloxy derivatives thereof (see, for example, Harris, et al., U.S. Pat. No. 5,252,714). The polymer may be branched or unbranched. Moreover, a mixture of polymers can be used to produce Zvn2R₁ conjugates.

Zvn2R₁ conjugates used for therapy can comprise pharmaceutically acceptable water-soluble polymer moieties. Suitable water-soluble polymers include polyethylene glycol (PEG), monomethoxy-PEG, mono-(C₁-C₁₀)alkoxy-PEG, aryloxy-PEG, poly-(N-vinyl pyrrolidone)PEG, tresyl monomethoxy PEG, PEG propionaldehyde, bis-succinimidyl carbonate PEG, propylene glycol homopolymers, a polypropylene oxide/ethylene oxide co-polymer, polyoxyethylated polyols (e.g., glycerol), polyvinyl alcohol, dextran, cellulose, or other carbohydrate-based polymers. Suitable PEG may have a molecular weight from about 600 to about 60,000, including, for example, 5,000, 12,000, 20,000 and 25,000. A Zvn2R1 conjugate can also comprise a mixture of such water-soluble polymers.

One example of a Zvn2R1 conjugate comprises a Zvn2R1 moiety and a polyalkyl oxide moiety attached to the N-terminus of the Zvn2R1 moiety. PEG is one suitable polyalkyl oxide. As an illustration, Zvn2R1 can be modified with PEG, a process known as “PEGylation.” PEGylation of Zvn2R1 can be carried out by any of the PEGylation reactions known in the art (see, for example, EP 0 154 316, Delgado et al., Critical Reviews in Therapeutic Drug Carrier Systems 9:249 (1992), Duncan and Spreafico, Clin. Pharmacokinet. 27:290 (1994), and Francis et al., Int J Hematol 68:1 (1998)). For example, PEGylation can be performed by an acylation reaction or by an alkylation reaction with a reactive polyethylene glycol molecule. In an alternative approach, Zvn2R1 conjugates are formed by condensing activated PEG, in which a terminal hydroxy or amino group of PEG has been replaced by an activated linker (see, for example, Karasiewicz et al., U.S. Pat. No. 5,382,657).

PEGylation by acylation typically requires reacting an active ester derivative of PEG with a Zvn2R1 polypeptide. An example of an activated PEG ester is PEG esterified to N-hydroxysuccinimide. As used herein, the term “acylation” includes the following types of linkages between Zvn2R1 and a water-soluble polymer: amide, carbamate, urethane, and the like. Methods for preparing PEGylated Zvn2R1 by acylation will typically comprise the steps of (a) reacting a Zvn2R1 polypeptide with PEG (such as a reactive ester of an aldehyde derivative of PEG) under conditions whereby one or more PEG groups attach to Zvn2R1, and (b) obtaining the reaction product(s). Generally, the optimal reaction conditions for acylation reactions will be determined based upon known parameters and desired results. For example, the larger the ratio of PEG:Zvn2R1, the greater the percentage of polyPEGylated Zvn2R1 product.

The product of PEGylation by acylation is typically a polyPEGylated Zvn2R1 product, wherein the lysine ε-amino groups are PEGylated via an acyl linking group. An example of a connecting linkage is an amide. Typically, the resulting Zvn2R1 will be at least 95% mono-, di-, or tri-pegylated, although some species with higher degrees of PEGylation may be formed depending upon the reaction conditions. PEGylated species can be separated from unconjugated Zvn2R1 polypeptides using standard purification methods, such as dialysis, ultrafiltration, ion exchange chromatography, affinity chromatography, and the like.

PEGylation by alkylation generally involves reacting a terminal aldehyde derivative of PEG with Zvn2R1 in the presence of a reducing agent. PEG groups can be attached to the polypeptide via a -CH₂-NH group.

Derivatization via reductive alkylation to produce a monoPEGylated product takes advantage of the differential reactivity of different types of primary amino groups available for derivatization. Typically, the reaction is performed at a pH that allows one to take advantage of the pKa differences between the ε-amino groups of the lysine residues and the α-amino group of the N-terminal residue of the protein. By such selective derivatization, attachment of a water-soluble polymer that contains a reactive group such as an aldehyde, to a protein is controlled. The conjugation with the polymer occurs predominantly at the N-terminus of the protein without significant modification of other reactive groups such as the lysine side chain amino groups. The present invention provides a substantially homogenous preparation of Zvn2R1 monopolymer conjugates.

Reductive alkylation to produce a substantially homogenous population of monopolymer Zvn2R1 conjugate molecule can comprise the steps of: (a) reacting a Zvn2R1 polypeptide with a reactive PEG under reductive alkylation conditions at a pH suitable to permit selective modification of the α-amino group at the amino terminus of the Zvn2R1, and (b) obtaining the reaction product(s). The reducing agent used for reductive alkylation should be stable in aqueous solution and able to reduce only the Schiff base formed in the initial process of reductive alkylation. Illustrative reducing agents include sodium borohydride, sodium cyanoborohydride, dimethylamine borane, trimethylamine borane, and pyridine borane.

For a substantially homogenous population of monopolymer Zvn2R1 conjugates, the reductive alkylation reaction conditions are those which permit the selective attachment of the water soluble polymer moiety to the N-terminus of Zvn2R1. Such reaction conditions generally provide for pKa differences between the lysine amino groups and the α-amino group at the N-terrninus. The pH also affects the ratio of polymer to protein to be used. In general, if the pH is lower, a larger excess of polymer to protein will be desired because the less reactive the N-terminal α-group, the more polymer is needed to achieve optimal conditions. If the pH is higher, the polymer:Zvn2R1 need not be as large because more reactive groups are available. Typically, the pH will fall within the range of 3 to 9, or 3 to 6.

Another factor to consider is the molecular weight of the water-soluble polymer. Generally, the higher the molecular weight of the polymer, the fewer number of polymer molecules which may be attached to the protein. For PEGylation reactions, the typical molecular weight is about 2 kDa to about 100 kDa, about 5 kDa to about 50 kDa, or about 12 kDa to about 25 kDa. The molar ratio of water-soluble polymer to Zvn2R1 will generally be in the range of 1:1 to 100:1. Typically, the molar ratio of water-soluble polymer to Zvn2R1 will be 1:1 to 20:1 for polyPEGylation, and 1:1 to 5:1 for monoPEGylation.

General methods for producing conjugates comprising a polypeptide and water-soluble polymer moieties are known in the art. See, for example, Karasiewicz et al., U.S. Pat. No. 5,382,657, Greenwald et al., U.S. Pat. No. 5,738, 846, Nieforth et al., Clin. Pharmacol. Ther. 59:636 (1996), Monkarsh et al., Anal. Biochem. 247:434 (1997)).

The present invention contemplates compositions comprising a peptide or polypeptide described herein. Such compositions can further comprise a carrier. The carrier can be a conventional organic or inorganic carrier. Examples of carriers include water, buffer solution, alcohol, propylene glycol, macrogol, sesame oil, corn oil, and the like.

7. Identification of Zvn2R1 Ligands

Zvn2R1 polypeptides can be used to identify and to isolate molecules that bind Zvn2R1 (“Zvn2R1 ligands”). For example, cells that produce Zvn2R1 proteins and peptides, or membrane preparations of such cells, can be used to bind ligands from a biological sample.

The activity of a Zvn2R1 polypeptide can be observed by a silicon-based biosensor microphysiometer, which measures the extracellular acidification rate or proton excretion associated with receptor binding and subsequent physiologic cellular responses. An exemplary device is the CYTOSENSOR Microphysiometer manufactured by Molecular Devices, Sunnyvale, Calif. A variety of cellular responses, such as cell proliferation, ion transport, energy production, inflammatory response, regulatory and receptor activation, and the like, can be measured by this method (see, for example, McConnell et al., Science 257:1906 (1992), Pitchford et al., Meth. Enzymol. 228:84 (1997), Arimilli et al., J. Immunol. Meth. 212:49 (1998), Van Liefde et al., Eur. J. Pharmacol. 346:87 (1998)). The microphysiometer can be used for assaying eukaryotic, prokaryotic, adherent, or non-adherent cells. By measuring extracellular acidification changes in cell media over time, the microphysiometer directly measures cellular responses to various stimuli, including naturally occurring ligands of Zvn2R1, Zvn2R1 agonists, or Zvn2R1 antagonists.

The microphysiometer can be used to measure responses of a Zvn2R1-expressing eukaryotic cell, compared to a control eukaryotic cell that does not produce Zvn2R1 polypeptide. Suitable cells responsive to Zvn2R1-modulating stimuli include recombinant host cells comprising a Zvn2R1 expression vector, and cells that naturally produce Zvn2R1. Extracellular acidification provides one measure for a Zvn2R1-modulated cellular response. In addition, this approach can be used to identify ligands, Zvn2R1 agonists, and Zvn2R1 antagonists. For example, a molecule can be identified as a Zvn2R1 agonist by providing cells that produce a Zvn2R1 polypeptide, culturing a first portion of the cells in the absence of the test compound, culturing a second portion of the cells in the presence of the test compound, and determining whether the second portion exhibits a cellular response, in comparison with the first portion.

Alternatively, a solid phase system can be used to identify a Zvn2R1 ligand, a Zvn2R1 agonist, or Zvn2R1 antagonist. For example, a cell that produces a Zvn2R1 polypeptide or Zvn2R1 fusion protein can be immobilized onto the surface of a receptor chip of a commercially available biosensor instrument (BLACORE, Biacore AB; Uppsala, Sweden). The use of this instrument is disclosed, for example, by Karlsson, Immunol. Methods 145:229 (1991), and Cunningham and Wells, J. Mol. Biol. 234:554 (1993).

In brief, cells that produce Zvn2R1 polypeptide or fusion protein, or membrane preparations comprising Zvn2R1 polypeptides, are covalently attached to dextran fibers that are attached to gold film within a flow cell. A test sample is then passed through the cell. If a ligand is present in the sample, it will bind to the immobilized polypeptide or fusion protein, causing a change in the refractive index of the medium, which is detected as a change in surface plasmon resonance of the gold film. This system allows the determination of on- and off-rates, from which binding affinity can be calculated, and assessment of stoichiometry of binding. This system can also be used to examine antibody-antigen interactions, and the interactions of other complement/anti-complement pairs. A suitable polypeptide for identifying Zvn2R1-binding molecules is a polypeptide comprising amino acid residues 30 to 610 of SEQ ID NO:8.

In another solid phase approach, Zvn2R1 polypeptides (e.g., amino acid sequences comprising amino acid residues 30 to 610 of SEQ ID NO:8) can be immobilized on a chromatography support to isolate Zvn2R1-binding molecules from a test sample, which is passed through the support. Suitable polypeptides for this approach include a fusion protein comprising an Fc moiety and an amino acid sequence comprising amino acid residues 30 to 610 of SEQ ID NO:8. General methods for performing affinity chromatography are well-known to those of skill in the art. See, for example, Affinity Chromatography:Principles & Methods (Pharmacia LKB Biotechnology 1988), and Doonan, Protein Purification Protocols (The Humana Press 1996).

Those of skill in the art are aware of additional methods for detecting the binding of putative ligands to membrane-bound receptors, such as the use of cellular screening systems with reporter genes, and the use of solubilized receptors. See, for example, Mills and Duggan, Trends Biotechnol. 12:47 (1994), Keen, M. (Ed.), Receptor Binding Techniques (Humana Press, Inc. 1998), Strosberg and Fernandes, “Recombinant G-Protein-coupled receptors as screening tools for drug discovery,” in Leff, P. (Ed.), Receptor-Based Drug Design (Marcel Dekker, Inc. 1998), Benovic, J. L., Regulation of G Protein Coupled Receptor Function and Expression: Receptor Biochemistry and Methodology (Wiley-Liss, 1999), Krieger et al., J. Biol. Chem. 274:4655 (1999), and Lynch, K. R. (Ed.), Identification and Expression of G Protein-Coupled Receptors (Wiley-Liss, 1999).

Ligand-binding domains of Zvn2R1 can be further characterized by physical analysis of structure, as determined by such techniques as nuclear magnetic resonance, crystallography, electron diffraction or photoaffinity labeling, in conjunction with mutation of putative contact site amino acids of Zvn2R1 ligand agonists. See, for example, de Vos et al., Science 255:306 (1992), Smith et al., J. Mol. Biol. 224:899 (1992), and Wlodaver et al., FEBS Lett. 309:59 (1992).

8. Production of Antibodies to Zvn2R1 Proteins

Antibodies to Zvn2R1 can be obtained, for example, using the product of a Zvn2R1 expression vector or Zvn2R1 isolated from a natural source as an antigen. Particularly useful anti-Zvn2R1 antibodies “bind specifically” with Zvn2R1. Antibodies are considered to be specifically binding if the antibodies exhibit at least one of the following two properties: (1) antibodies bind to Zvn2R1 with a threshold level of binding activity, and (2) antibodies do not significantly cross-react with polypeptides related to Zvn2R1.

With regard to the first characteristic, antibodies specifically bind if they bind to a Zvn2R1 polypeptide, peptide or epitope with a binding affinity (Kₐ) of 10⁶M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660 (1949)). With regard to the second characteristic, antibodies do not significantly cross-react with related polypeptide molecules, for example, if they detect Zvn2R1, but not presently known polypeptides using a standard Western blot analysis. Examples of known related polypeptides include known vomeronasal receptors, such as the murine tissue-type vomeronasal pheromone receptor V2R2 (GenBank Accession No. AF053986).

Anti-Zvn2R1 antibodies can be produced using antigenic Zvn2R1 epitope-bearing peptides and polypeptides. Antigenic epitope-bearing peptides and polypeptides of the present invention contain a sequence of at least nine, or between 15 to about 30 amino acids contained within SEQ ID NO:2 or another amino acid sequence disclosed herein. However, peptides or polypeptides comprising a larger portion of an amino acid sequence of the invention, containing from 30 to 50 amino acids, or any length up to and including the entire amino acid sequence of a polypeptide of the invention, also are useful for inducing antibodies that bind with Zvn2R1. It is desirable that the amino acid sequence of the epitope-bearing peptide is selected to provide substantial solubility in aqueous solvents (i.e., the sequence includes relatively hydrophilic residues, while hydrophobic residues are preferably avoided). Moreover, amino acid sequences containing proline residues may be also be desirable for antibody production.

As an illustration, potential antigenic sites in Zvn2R1 were identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

The Jameson-Wolf method predicts potential antigenic determinants by combining six major subroutines for protein structural prediction. Briefly, the Hopp-Woods method, Hopp et al., Proc. Nat'l Acad. Sci. USA 78:3824 (1981), was first used to identify amino acid sequences representing areas of greatest local hydrophilicity (parameter: seven residues averaged). In the second step, Emini's method, Emini et al., J. Virology 55:836 (1985), was used to calculate surface probabilities (parameter: surface decision threshold (0.6)=1). Third, the Karplus-Schultz method, Karplus and Schultz, Naturwissenschaften 72:212 (1985), was used to predict backbone chain flexibility (parameter: flexibility threshold (0.2)=1). In the fourth and fifth steps of the analysis, secondary structure predictions were applied to the data using the methods of Chou-Fasman, Chou, “Prediction of Protein Structural Classes from Amino Acid Composition,” in Prediction of Protein Structure and the Principles of Protein Conformation, Fasman (ed.), pages 549-586 (Plenum Press 1990), and Garnier-Robson, Gamier et al., J. Mol. Biol. 120:97 (1978) (Chou-Fasman parameters: conformation table=64 proteins; a region threshold=103; β region threshold=105; Garnier-Robson parameters:α and ↑ decision constants=0). In the sixth subroutine, flexibility parameters and hydropathy/solvent accessibility factors were combined to determine a surface contour value, designated as the “antigenic index.” Finally, a peak broadening function was applied to the antigenic index, which broadens major surface peaks by adding 20, 40, 60, or 80% of the respective peak value to account for additional free energy derived from the mobility of surface regions relative to interior regions. This calculation was not applied, however, to any major peak that resides in a helical region, since helical regions tend to be less flexible.

The results of this analysis indicated that the following amino acid sequences would provide suitable antigenic molecules: amino acids 26 to 39 of SEQ ID NO:2 (“antigenic molecule 1”), amino acids 61 to 71 of SEQ ID NO:2 (“antigenic molecule 2”), amino acids 73 to 81 of SEQ ID NO:2 (“antigenic molecule 3”), amino acids 61 to 81 of SEQ ID NO:2 (“antigenic molecule 4”), amino acids 100 to 109 of SEQ ID NO:2 (“antigenic molecule 5”), amino acids 137 to 150 of SEQ ID NO:2 (“antigenic molecule 6”), amino acids 37 to 53 of SEQ ID NO:5 (“antigenic molecule 7”), amino acids 36 to 61 of SEQ ID NO:5 (“antigenic molecule 8”), and amino acids 354 to 368 of SEQ ID NO:5 (“antigenic molecule 9”). The present invention contemplates the use of any one of antigenic molecules 1 to 9 to generate antibodies to Zvn2R1. The present invention also contemplates polypeptides comprising at least one of antigenic molecules 1 to 9.

In addition, antibodies can be directed against extracellular or intracellular domains of Zvn2R1. These domains are identified in Table 1.

Polyclonal antibodies to recombinant Zvn2R1 protein or to Zvn2R1 isolated from natural sources can be prepared using methods well-known to those of skill in the art. See, for example, Green et al., “Production of Polyclonal Antisera,” in Immunochemical Protocols (Manson, ed.), pages 1-5 (Humana Press 1992), and Williams et al., “Expression of foreign proteins in E. coli using plasmid vectors and purification of specific polyclonal antibodies,” in DNA Cloning 2:Expression Systems, 2nd Edition, Glover et al. (eds.), page 15 (Oxford University Press 1995). The immunogenicity of a Zvn2R1 polypeptide can be increased through the use of an adjuvant, such as alum (aluminum hydroxide) or Freund's complete or incomplete adjuvant. Polypeptides useful for immunization also include fusion polypeptides, such as fusions of Zvn2R1 or a portion thereof with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, guinea pigs, goats, or sheep, an anti-Zvn2R1 antibody of the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310 (1990).

Alternatively, monoclonal anti-Zvn2R1 antibodies can be generated. Rodent monoclonal antibodies to specific antigens may be obtained by methods known to those skilled in the art (see, for example, Kohler et al., Nature 256:495 (1975), Coligan et al. (eds.), Current Protocols in Immunology, Vol. 1, pages 2.5.1-2.6.7 (John Wiley & Sons 1991) [“Coligan”], Picksley et al., “Production of monoclonal antibodies against proteins expressed in E. coli,” in DNA Cloning 2:Expression Systems, 2nd Edition, Glover et al. (eds.), page 93 (Oxford University Press 1995)).

Briefly, monoclonal antibodies can be obtained by injecting mice with a composition comprising a Zvn2R1 gene product, verifying the presence of antibody production by removing a serum sample, removing the spleen to obtain B-lymphocytes, fusing the B-lymphocytes with myeloma cells to produce hybridomas, cloning the hybridomas, selecting positive clones that produce antibodies to the antigen, culturing the clones that produce antibodies to the antigen, and isolating the antibodies from the hybridoma cultures.

In addition, an anti-Zvn2R1 antibody of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13 (1994), Lonberg et al., Nature 368:856 (1994), and Taylor et al., Int. Immun. 6:579 (1994).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)).

For particular uses, it may be desirable to prepare fragments of anti-Zvn2R1 antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 89:230 (1960), Porter, Biochem. J. 73:119 (1959), Edelman et al., in Methods in Enzymology Vol. 1, page 422 (Academic Press 1967), and by Coligan at pages 2.8.1-2.8.10 and 2.10.-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Nat'l Acad. Sci. USA 69:2659 (1972). Alternatively, the variable chains can be linked by an intermolecular disulfide bond or cross-linked by chemicals such as glutaraldehyde (see, for example, Sandhu, Crit. Rev. Biotech. 12:437 (1992)).

The Fv fragments may comprise V_{H} and V_{L} chains, which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing scFvs are described, for example, by Whitlow et al., Methods:A Companion to Methods in Enzymology 2:97 (1991) (also see, Bird et aL, Science 242:423 (1988), Ladner et al., U.S. Pat. No. 4,946,778, Pack et al., Bio/Technology 11: 1271 (1993), and Sandhu, supra).

As an illustration, a scFV can be obtained by exposing lymphocytes to Zvn2R1 polypeptide in vitro, and selecting antibody display libraries in phage or similar vectors (for instance, through use of immobilized or labeled Zvn2R1 protein or peptide). Genes encoding polypeptides having potential Zvn2R1 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides, which interact with a known target, which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from CLONTECH Laboratories, Inc. (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the Zvn2R1 sequences disclosed herein to identify proteins, which bind to Zvn2R1.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods:A Companion to Methods in Enzymology 2:106 (1991), Courtenay-Luck, “Genetic Manipulation of Monoclonal Antibodies,” in Monoclonal Antibodies:Production, Engineering and Clinical Application, Ritter et al. (eds.), page 166 (Cambridge University Press 1995), and Ward et al., “Genetic Manipulation and Expression of Antibodies,” in Monoclonal Antibodies:Principles and Applications, Birch et al., (eds.), page 137 (Wiley-Liss, Inc. 1995)).

Alternatively, an anti-Zvn2R1 antibody may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989). Techniques for producing humanized monoclonal antibodies are described, for example, by Jones et al., Nature 321:522 (1986), Carter et al., Proc. Nat'l Acad. Sci. USA 89:4285 (1992), Sandhu, Crit. Rev. Biotech. 12:437 (1992), Singer et al., J. Immun. 150:2844 (1993), Sudhir (ed.), Antibody Engineering Protocols (Humana Press, Inc. 1995), Kelley, “Engineering Therapeutic Antibodies,” in Protein Engineering:Principles and Practice, Cleland et al. (eds.), pages 399-434 (John Wiley & Sons, Inc. 1996), and by Queen et al., U.S. Pat. No. 5,693,762 (1997).

Polyclonal anti-idiotype antibodies can be prepared by immunizing animals with anti-Zvn2R1 antibodies or antibody fragments, using standard techniques. See, for example, Green et al., “Production of Polyclonal Antisera,” in Methods In Molecular Biology:Immunochemical Protocols, Manson (ed.), pages 1-12 (Humana Press 1992). Also, see Coligan at pages 2.4.1-2.4.7. Alternatively, monoclonal anti-idiotype antibodies can be prepared using anti-Zvn2R1 antibodies or antibody fragments as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677, and Varthakavi and Minocha, J. Gen. Virol. 77:1875 (1996).

9. Use of Zvn2R1 Nucleotide Sequences to Detect Gene Expression and Gene Structure

Nucleic acid molecules can be used to detect the expression of a Zvn2R1 gene in a biological sample. Certain probe molecules include double-stranded nucleic acid molecules comprising the nucleotide sequence of SEQ ID NOs:1 or 4, or a portion thereof, as well as single-stranded nucleic acid molecules having the complement of the nucleotide sequence of SEQ ID NOs:1 or 4, or a portion thereof. Probe molecules may be DNA, RNA, oligonucleotides, and the like. As used herein, the term “portion” refers to at least eight nucleotides to at least 20 or more nucleotides. Certain probes bind with regions of the Zvn2R1 gene that have a low sequence similarity to comparable regions in other vomeronasal receptor genes.

In a basic assay, a single-stranded probe molecule is incubated with RNA, isolated from a biological sample, under conditions of temperature and ionic strength that promote base pairing between the probe and target Zvn2R1 RNA species. After separating unbound probe from hybridized molecules, the amount of hybrids is detected.

Well-established hybridization methods of RNA detection include northern analysis and dot/slot blot hybridization (see, for example, Ausubel (1995) at pages 4-1 to 4-27, and Wu et al. (eds.), “Analysis of Gene Expression at the RNA Level,” in Methods in Gene Biotechnology, pages 225-239 (CRC Press, Inc. 1997)). Nucleic acid probes can be detectably labeled with radioisotopes such as ³²P or ³⁵S. Alternatively, Zvn2R1 RNA can be detected with a nonradioactive hybridization method (see, for example, Isaac (ed.), Protocols for Nucleic Acid Analysis by Nonradioactive Probes (Humana Press, Inc. 1993)). Typically, nonradioactive detection is achieved by enzymatic conversion of chromogenic or chemiluminescent substrates. Illustrative nonradioactive moieties include biotin, fluorescein, and digoxigenin.

Zvn2R1 oligonucleotide probes are also useful for in vivo diagnosis. As an illustration, ¹⁸F-labeled oligonucleotides can be administered to a subject and visualized by positron emission tomography (Tavitian et al., Nature Medicine 4:467 (1998)).

Numerous diagnostic procedures take advantage of the polymerase chain reaction (PCR) to increase sensitivity of detection methods. Standard techniques for performing PCR are well-known (see, generally, Mathew (ed.), Protocols in Human Molecular Genetics (Humana Press, Inc. 1991), White (ed.), PCR Protocols:Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)).

PCR primers can be designed to amplify a portion of the Zvn2R1 gene that has a low sequence similarity to a comparable region in other proteins, such as other vomeronasal receptor proteins.

One variation of PCR for diagnostic assays is reverse transcriptase-PCR (RT-PCR). In the RT-PCR technique, RNA is isolated from a biological sample, reverse transcribed to cDNA, and the cDNA is incubated with Zvn2R1 primers (see, for example, Wu et al. (eds.), “Rapid Isolation of Specific cDNAs or Genes by PCR,” in Methods in Gene Biotechnology, pages 15-28 (CRC Press, Inc. 1997)). PCR is then performed and the products are analyzed using standard techniques.

As an illustration, RNA is isolated from biological sample using, for example, the guanidinium-thiocyanate cell lysis procedure described above. Alternatively, a solid-phase technique can be used to isolate mRNA from a cell lysate. A reverse transcription reaction can be primed with the isolated RNA using random oligonucleotides, short homopolymers of dT, or Zvn2R1 anti-sense oligomers. Oligo-dT primers offer the advantage that various mRNA nucleotide sequences are amplified that can provide control target sequences. Zvn2R1 sequences are amplified by the polymerase chain reaction using two flanking oligonucleotide primers that are typically 20 bases in length.

PCR amplification products can be detected using a variety of approaches. For example, PCR products can be fractionated by gel electrophoresis, and visualized by ethidium bromide staining. Alternatively, fractionated PCR products can be transferred to a membrane, hybridized with a detectably-labeled Zvn2R1 probe, and examined by autoradiography. Additional alternative approaches include the use of digoxigenin-labeled deoxyribonucleic acid triphosphates to provide chemiluminescence detection, and the C-TRAK colorimetric assay.

Another approach for detection of Zvn2R1 expression is cycling probe technology, in which a single-stranded DNA target binds with an excess of DNA-RNA-DNA chimeric probe to form a complex, the RNA portion is cleaved with RNAase H, and the presence of cleaved chimeric probe is detected (see, for example, Beggs et al., J. Clin. Microbiol. 34:2985 (1996), Bekkaoui et al., Biotechniques 20:240 (1996)). Alternative methods for detection of Zvn2R1 sequences can utilize approaches such as nucleic acid sequence-based amplification, cooperative amplification of templates by cross-hybridization, and the ligase chain reaction (see, for example, Marshall et al., U.S. Pat. No. 5,686,272 (1997), Dyer et al., J. Virol. Methods 60:161 (1996), Ehricht et al., Eur. J. Biochem. 243:358 (1997), and Chadwick et al., J. Virol. Methods 70:59 (1998)). Other standard methods are known to those of skill in the art.

Zvn2R1 probes and primers can also be used to detect and to localize Zvn2R1 gene expression in tissue samples. Methods for such in situ hybridization are well-known to those of skill in the art (see, for example, Choo (ed.), In Situ Hybridization Protocols (Humana Press, Inc. 1994), Wu et al. (eds.), “Analysis of Cellular DNA or Abundance of mRNA by Radioactive In Situ Hybridization (RISH),” in Methods in Gene Biotechnology, pages 259-278 (CRC Press, Inc. 1997), and Wu et al. (eds.), “Localization of DNA or Abundance of mRNA by Fluorescence In Situ Hybridization (RISH),” in Methods in Gene Biotechnology, pages 279-289 (CRC Press, Inc. 1997)). Various additional diagnostic approaches are well-known to those of skill in the art (see, for example, Mathew (ed.), Protocols in Human Molecular Genetics (Humana Press, Inc. 1991), Coleman and Tsongalis, Molecular Diagnostics (Humana Press, Inc. 1996), and Elles, Molecular Diagnosis of Genetic Diseases (Humana Press, Inc., 1996)). Suitable test samples include blood, urine, saliva, tissue biopsy, and autopsy material.

The chromosomal location of the Zvn2R1 gene can be identified using radiation hybrid mapping, which is a somatic cell genetic technique developed for constructing high-resolution, contiguous maps of mammalian chromosomes (Cox et al., Science 250:245 (1990)). Partial or full knowledge of a gene sequence allows one to design PCR primers suitable for use with chromosomal radiation hybrid mapping panels. Radiation hybrid mapping panels are commercially available which cover the entire human genome, such as the Stanford G3 RH Panel and the GeneBridge 4 RH Panel (Research Genetics, Inc., Huntsville, Ala.). These panels enable rapid, PCR-based chromosomal localizations and ordering of genes, sequence-tagged sites, and other nonpolymorphic and polymorphic markers within a region of interest. This includes establishing directly proportional physical distances between newly discovered genes of interest and previously mapped markers.

Nucleic acid molecules comprising Zvn2R1 nucleotide sequences can also be used to determine whether a subject's chromosomes contain a mutation in the Zvn2R1 gene. Detectable chromosomal aberrations at the Zvn2R1 gene locus include, but are not limited to, aneuploidy, gene copy number changes, insertions, deletions, restriction site changes and rearrangements. Of particular interest are genetic alterations that inactivate the Zvn2R1 gene.

Aberrations associated with the Zvn2R1 locus can be detected using nucleic acid molecules of the present invention by employing molecular genetic techniques, such as restriction fragment length polymorphism analysis, short tandem repeat analysis employing PCR techniques, amplification-refractory mutation system analysis, single-strand conformation polymorphism detection, RNase cleavage methods, denaturing gradient gel electrophoresis, fluorescence-assisted mismatch analysis, and other genetic analysis techniques known in the art (see, for example, Mathew (ed.), Protocols in Human Molecular Genetics (Humana Press, Inc. 1991), Marian, Chest 108:255 (1995), Coleman and Tsongalis, Molecular Diagnostics (Humana Press, Inc. 1996), Elles (ed.) Molecular Diagnosis of Genetic Diseases (Humana Press, Inc. 1996), Landegren (ed.), Laboratory Protocols for Mutation Detection (Oxford University Press 1996), Birren et al. (eds.), Genome Analysis, Vol. 2:Detecting Genes (Cold Spring Harbor Laboratory Press 1998), Dracopoli et al. (eds.), Current Protocols in Human Genetics (John Wiley & Sons 1998), and Richards and Ward, “Molecular Diagnostic Testing,” in Principles of Molecular Medicine, pages 83-88 (Humana Press, Inc. 1998)).

The protein truncation test is also useful for detecting the inactivation of a gene in which translation-terminating mutations produce only portions of the encoded protein (see, for example, Stoppa-Lyonnet et al., Blood 91:3920 (1998)). According to this approach, RNA is isolated from a biological sample, and used to synthesize cDNA. PCR is then used to amplify the Zvn2R1 target sequence and to introduce an RNA polymerase promoter, a translation initiation sequence, and an in-frame ATG triplet. PCR products are transcribed using an RNA polymerase, and the transcripts are translated in vitro with a T7-coupled reticulocyte lysate system. The translation products are then fractionated by SDS-PAGE to determine the lengths of the translation products. The protein truncation test is described, for example, by Dracopoli et al. (eds.), Current Protocols in Human Genetics, pages 9.11.1 - 9.11.18 (John Wiley & Sons 1998).

The present invention also contemplates kits for performing an assay for Zvn2R1 gene expression, or to detect mutations in the Zvn2R1 gene. Such kits comprise nucleic acid probes, such as double-stranded nucleic acid molecules comprising the nucleotide sequence of SEQ ID NOs:1 or 4, or a portion thereof, as well as single-stranded nucleic acid molecules having the complement of the nucleotide sequence of SEQ ID NOs:1 or 4, or a portion thereof. Probe molecules may be DNA, RNA, oligonucleotides, and the like. Kits may comprise nucleic acid primers for performing PCR.

Such a kit can contain all the necessary elements to perform a nucleic acid diagnostic assay described above. A kit will comprise at least one container comprising a Zvn2R1 probe or primer. The kit may also comprise a second container comprising one or more reagents capable of indicating the presence of Zvn2R1 sequences. Examples of such indicator reagents include detectable labels such as radioactive labels, fluorochromes, chemiluminescent agents, and the like. A kit may also comprise a means for conveying to the user that the Zvn2R1 probes and primers are used to detect Zvn2R1 gene expression. For example, written instructions may state that the enclosed nucleic acid molecules can be used to detect either a nucleic acid molecule that encodes Zvn2R1, or a nucleic acid molecule having a nucleotide sequence that is complementary to a Zvn2R1-encoding nucleotide sequence. The written material can be applied directly to a container, or the written material can be provided in the form of a packaging insert.

10. Use of Anti-Zvn2R1 Antibodies to Detect Zvn2R1

The present invention contemplates the use of anti-Zvn2R1 antibodies to screen biological samples in vitro for the presence of Zvn2R1. In one type of in vitro assay, anti-Zvn2R1 antibodies are used in liquid phase. For example, the presence of Zvn2R1 in a biological sample can be tested by mixing the biological sample with a trace amount of labeled Zvn2R1 and an anti-Zvn2R1 antibody under conditions that promote binding between Zvn2R1 and its antibody. Complexes of Zvn2R1 and anti-Zvn2R1 in the sample can be separated from the reaction mixture by contacting the complex with an immobilized protein which binds with the antibody, such as an Fc antibody or Staphylococcus protein A. The concentration of Zvn2R1 in the biological sample will be inversely proportional to the amount of labeled Zvn2R1 bound to the antibody and directly related to the amount of free-labeled Zvn2R1. Illustrative biological samples include blood, urine, saliva, tissue biopsy, and autopsy material.

Alternatively, in vitro assays can be performed in which anti-Zvn2R1 antibody is bound to a solid-phase carrier. For example, antibody can be attached to a polymer, such as aminodextran, in order to link the antibody to an insoluble support such as a polymer-coated bead, a plate or a tube. Other suitable in vitro assays will be readily apparent to those of skill in the art.

In another approach, anti-Zvn2R1 antibodies can be used to detect Zvn2R1 in tissue sections prepared from a biopsy specimen. Such immunochemical detection can be used to determine the relative abundance of Zvn2R1 and to determine the distribution of Zvn2R1 in the examined tissue. General immunochemistry techniques are well established (see, for example, Ponder, “Cell Marking Techniques and Their Application,” in Mammalian Development:A Practical Approach, Monk (ed.), pages 115-38 (IRL Press 1987), Coligan at pages 5.8.1-5.8.8, Ausubel (1995) at pages 14.6.1 to 14.6.13 (Wiley Interscience 1990), and Manson (ed.), Methods In Molecular Biology, Vol. 10:Immunochemical Protocols (The Humana Press, Inc. 1992)).

Immunochemical detection can be performed by contacting a biological sample with an anti-Zvn2R1 antibody, and then contacting the biological sample with a detectably labeled molecule, which binds to the antibody. For example, the detectably labeled molecule can comprise an antibody moiety that binds to anti-Zvn2R1 antibody. Alternatively, the anti-Zvn2R1 antibody can be conjugated with avidin/streptavidin (or biotin) and the detectably labeled molecule can comprise biotin (or avidinlstreptavidin). Numerous variations of this basic technique are well-known to those of skill in the art.

Alternatively, an anti-Zvn2R1 antibody can be conjugated with a detectable label to form an anti-Zvn2R1 immunoconjugate. Suitable detectable labels include, for example, a radioisotope, a fluorescent label, a chemiluminescent label, an enzyme label, a bioluminescent label or colloidal gold. Methods of making and detecting such detectably-labeled immunoconjugates are well-known to those of ordinary skill in the art, and are described in more detail below.

The detectable label can be a radioisotope that is detected by autoradiography. Isotopes that are particularly useful for the purpose of the present invention are ³H, ¹²⁵I, ¹³¹I, ³⁵S and ¹⁴C.

Anti-Zvn2R¹ immunoconjugates can also be labeled with a fluorescent compound. The presence of a fluorescently-labeled antibody is determined by exposing the immunoconjugate to light of the proper wavelength and detecting the resultant fluorescence. Fluorescent labeling compounds include fluorescein isothiocyanate, rhoda-mine, phycoerytherin, phycocyanin, allophycocyanin, o-phthaldehyde and fluorescamine.

Alternatively, anti-Zvn2R1 immunoconjugates can be detectably labeled by coupling an antibody component to a chemiluminescent compound. The presence of the chemiluminescent-tagged immunoconjugate is determined by detecting the presence of luminescence that arises during the course of a chemical reaction. Examples of chemi-luminescent labeling compounds include luminol, isoluminol, an aromatic acridinium ester, an imidazole, an acridinium salt and an oxalate ester.

Similarly, a bioluminescent compound can be used to label anti-Zvn2R1 immunoconjugates of the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, anti-Zvn2R1 immunoconjugates can be detectably labeled by linking an anti-Zvn2R1 antibody component to an enzyme. When the anti-Zvn2R1-enzyme conjugate is incubated in the presence of the appropriate substrate, the enzyme moiety reacts with the substrate to produce a chemical moiety, which can be detected, for example, by spectrophotometric, fluorometric or visual means. Examples of enzymes that can be used to detectably label polyspecific immunoconjugates include β-galactosidase, glucose oxidase, peroxidase and alkaline phosphatase.

Those of skill in the art will know of other suitable labels, which can be employed in accordance with the present invention. The binding of marker moieties to anti-Zvn2R1 antibodies can be accomplished using standard techniques known to the art. Typical methodology in this regard is described by Kennedy et al., Clin. Chim. Acta 70:1 (1976), Schurs et al., Clin. Chim. Acta 81:1 (1977), Shih et al., Int'l J. Cancer 46:1101 (1990), Stein etal., Cancer Res. 50:1330 (1990), and Coligan, supra.

Moreover, the convenience and versatility of immunochernical detection can be enhanced by using anti-Zvn2R1 antibodies that have been conjugated with avidin, streptavidin, and biotin (see, for example, Wilchek et al. (eds.), “Avidin-Biotin Technology,” Methods In Enzymology, Vol. 184 (Academic Press 1990), and Bayer et al., “Immunochemical Applications of Avidin-Biotin Technology,” in Methods In Molecular Biology, Vol. 10, Manson (ed.), pages 149-162 (The Humana Press, Inc. 1992).

Methods for performing immunoassays are well-established. See, for example, Cook and Self, “Monoclonal Antibodies in Diagnostic Immunoassays,” in Monoclonal Antibodies:Production, Engineering, and Clinical Application, Ritter and Ladyman (eds.), pages 180-208, (Cambridge University Press, 1995), Perry, “The Role of Monoclonal Antibodies in the Advancement of Inmunoassay Technology,” in Monoclonal Antibodies:Principles and Applications, Birch and Lennox (eds.), pages 107-120 (Wiley-Liss, Inc. 1995), and Diamandis, Immunoassay (Academic Press, Inc. 1996).

The present invention also contemplates kits for performing an immunological diagnostic assay for Zvn2R1 gene expression. Such kits comprise at least one container comprising an anti-Zvn2R1 antibody, or antibody fragment. A kit may also comprise a second container comprising one or more reagents capable of indicating the presence of Zvn2R1 antibody or antibody fragments. Examples of such indicator reagents include detectable labels such as a radioactive label, a fluorescent label, a chemiluminescent label, an enzyme label, a bioluminescent label, colloidal gold, and the like. A kit may also comprise a means for conveying to the user that Zvn2R1 antibodies or antibody fragments are used to detect Zvn2R1 protein. For example, written instructions may state that the enclosed antibody or antibody fragment can be used to detect Zvn2R1. The written material can be applied directly to a container, or the written material can be provided in the form of a packaging insert.

In addition to the detection kits described above, polynucleotides and polypeptides of the present invention will be useful as educational tools in laboratory practicum kits for courses related to genetics and molecular biology, protein chemistry, and antibody production and analysis. Due to its unique polynucleotide and polypeptide sequences, molecules of Zvn2R1 can be used as standards or as “unknowns” for testing purposes. For example, Zvn2R1 polynucleotides can be used as an aid, such as, for example, to teach a student how to prepare expression constructs for bacterial, viral, or mammalian expression, including fusion constructs, wherein Zvn2R1 is the gene to be expressed; for determining the restriction endonuclease cleavage sites of the polynucleotides; determining MRNA and DNA localization of Zvn2R1 polynucleotides in tissues (i.e., by northern and Southern blotting as well as polymerase chain reaction); and for identifying related polynucleotides and polypeptides by nucleic acid hybridization. As an illustration, students will find that Bg/II digestion of a nucleic acid molecule consisting of the nucleotide sequence of SEQ ID NO:1 provides fragments of 309 base pairs, and 348 base pairs, and that digestion with EcoRI provides fragments of 147 base pairs, and 510 base pairs.

Zvn2R1 polypeptides can be used as an aid to teach preparation of antibodies; identifying proteins by western blotting; protein purification; determining the weight of produced Zvn2R1 polypeptides as a ratio to total protein produced; identifying peptide cleavage sites; coupling amino and carboxyl terminal tags; amino acid sequence analysis, as well as, but not limited to monitoring biological activities of both the native and tagged protein (i.e., protease inhibition) in vitro and in vivo. For example, students will find that digestion of an unglycosylated Zvn2R1 polypeptide consisting of the amino acid sequence of SEQ ID NO:2 with BNPS or NCS/urea yields three fragments (approximate molecular weights:2735, 7624, and 14379), whereas digestion of such a polypeptide with NTCB provides six fragments (approximate molecular weights:4552, 688, 3664, 5047, 6857, and 3981).

Zvn2R1 polypeptides can also be used to teach analytical skills such as mass spectrometry, circular dichroism to determine conformation, especially of the four alpha helices, x-ray crystallography to determine the three-dimensional structure in atomic detail, nuclear magnetic resonance spectroscopy to reveal the structure of proteins in solution. For example, a kit containing the Zvn2R1 can be given to the student to analyze. Since the amino acid sequence would be known by the instructor, the protein can be given to the student as a test to determine the skills or develop the skills of the student, the instructor would then know whether or not the student has correctly analyzed the polypeptide. Since every polypeptide is unique, the educational utility of Zvn2R1 would be unique unto itself.

The antibodies which bind specifically to Zvn2R1 can be used as a teaching aid to instruct students how to prepare affinity chromatography columns to purify Zvn2R1, cloning and sequencing the polynucleotide that encodes an antibody and thus as a practicum for teaching a student how to design humanized antibodies. The Zvn2R1 gene, polypeptide, or antibody would then be packaged by reagent companies and sold to educational institutions so that the students gain skill in art of molecular biology. Because each gene and protein is unique, each gene and protein creates unique challenges and learning experiences for students in a lab practicum. Such educational kits containing the Zvn2R1 gene, polypeptide, or antibody are considered within the scope of the present invention.

11. Production of Transgenic Mice

Transgenic mice can be engineered to over-express the Zvn2R1 gene in all tissues or under the control of a tissue-specific or tissue-preferred regulatory element. These over-producers of Zvn2R1 can be used to characterize the phenotype that results from over-expression, and the transgenic animals can serve as models for human disease caused by excess Zvn2R1. Transgenic mice that over-express Zvn2R1 also provide model bioreactors for production of Zvn2R1 in the milk or blood of larger animals. Methods for producing transgenic mice are well-known to those of skill in the art (see, for example, Jacob, “Expression and Knockout of Interferons in Transgenic Mice,” in Overexpression and Knockout of Vomeronasals in Transgenic Mice, Jacob (ed.), pages 111-124 (Academic Press, Ltd. 1994), Monastersky and Robl (eds.), Strategies in Transgenic Animal Science (ASM Press 1995), and Abbud and Nilson, “Recombinant Protein Expression in Transgenic Mice,” in Gene Expression Systems:Using Nature for the Art of Expression, Fernandez and Hoeffler (eds.), pages 367-397 (Academic Press, Inc. 1999)).

Moreover, transgenic animals expressing the human Zvn2R1 transgene in their vomeronasal organ or in their olfactory epithelium may provide an in vivo assay model for the identification of a ligand for Zvn2R1 or as an experimental animal model system to study the effect of the human receptor ligand on physiology and behavior. Human Zvn2R1 expression can be directed to the vomeronasal organ or olfactory epithelium of transgenetic mice by the use or promoter elements derived from the murine olfactory receptor genes or by gene targeting (Rodriguez et aL, Cell 97:199 (1999); Belluscio et al., Cell 97:209 (1999)).

For example, a method for producing a transgenic mouse that expresses a Zvn2R1 gene can begin with adult, fertile males (studs) (B6C3f1, 2-8 months of age (Taconic Farms, Germantown, N.Y.)), vasectomized males (duds) (B6D2f1, 2-8 months, (Taconic Farms)), prepubescent fertile females (donors) (B6C3f1, 4-5 weeks, (Taconic Farms)) and adult fertile females (recipients) (B6D2f1, 2-4 months, (Taconic Farms)). The donors are acclimated for one week and then injected with approximately 8 IU/mouse of Pregnant Mare's Serum gonadotrophin (Sigma Chemical Company; St. Louis, Mo.) I.P., and 46-47 hours later, 8 IU/mouse of human Chorionic Gonadotropin (hCG (Sigma)) I.P. to induce superovulation. Donors are mated with studs subsequent to hormone injections. Ovulation generally occurs within 13 hours of hCG injection. Copulation is confirmed by the presence of a vaginal plug the morning following mating.

Fertilized eggs are collected under a surgical scope. The oviducts are collected and eggs are released into urinanalysis slides containing hyaluronidase (Sigma). Eggs are washed once in hyaluronidase, and twice in Whitten's W640 medium (described, for example, by Menino and O° Claray, Biol. Reprod. 77:159 (1986), and Dienhart and Downs, Zygote 4:129 (1996)) that has been incubated with 5% CO₂, 5% O₂, and 90% N₂ at 37° C. The eggs are then stored in a 37° C./5% CO₂ incubator until microinjection.

Ten to twenty micrograms of plasmid DNA containing a Zvn2R1 encoding sequence is linearized, gel-purified, and resuspended in 10 mM Tris-HCl (pH 7.4), 0.25 mM EDTA (pH 8.0), at a final concentration of 5-10 nanograms per microliter for microinjection. For example, the Zvn2R1 encoding sequences can encode a polypeptide comprising the amino acid sequence of SEQ ID NOs:2 or 5.

Plasmid DNA is microinjected into harvested eggs contained in a drop of W640 medium overlaid by warm, CO₂-equilibrated mineral oil. The DNA is drawn into an injection needle (pulled from a 0.75mm ID, 1 mm OD borosilicate glass capillary), and injected into individual eggs. Each egg is penetrated with the injection needle, into one or both of the haploid pronuclei.

Picoliters of DNA are injected into the pronuclei, and the injection needle withdrawn without coming into contact with the nucleoli. The procedure is repeated until all the eggs are injected. Successfully microinjected eggs are transferred into an organ tissue-culture dish with pre-gassed W640 medium for storage overnight in a 37° C./5% CO incubator.

The following day, two-cell embryos are transferred into pseudopregnant recipients. The recipients are identified by the presence of copulation plugs, after copulating with vasectomized duds. Recipients are anesthetized and shaved on the dorsal left side and transferred to a surgical microscope. A small incision is made in the skin and through the muscle wall in the middle of the abdominal area outlined by the ribcage, the saddle, and the hind leg, midway between knee and spleen. The reproductive organs are exteriorized onto a small surgical drape. The fat pad is stretched out over the surgical drape, and a baby serrefine (Roboz, Rockville, Md.) is attached to the fat pad and left hanging over the back of the mouse, preventing the organs from sliding back in.

With a fine transfer pipette containing mineral oil followed by alternating W640 and air bubbles, 12-17 healthy two-cell embryos from the previous day's injection are transferred into the recipient. The swollen ampulla is located and holding the oviduct between the ampulla and the bursa, a nick in the oviduct is made with a 28 g needle close to the bursa, making sure not to tear the ampulla or the bursa.

The pipette is transferred into the nick in the oviduct, and the embryos are blown in, allowing the first air bubble to escape the pipette. The fat pad is gently pushed into the peritoneum, and the reproductive organs allowed to slide in. The peritoneal wall is closed with one suture and the skin closed with a wound clip. The mice recuperate on a 37° C. slide warmer for a minimum of four hours.

The recipients are returned to cages in pairs, and allowed 19-21 days gestation. After birth, 19-21 days postpartum is allowed before weaning. The weanlings are sexed and placed into separate sex cages, and a 0.5 cm biopsy (used for genotyping) is snipped off the tail with clean scissors.

Genomic DNA is prepared from the tail snips using, for example, a QIAGEN DNEASY kit following the manufacturer's instructions. Genomic DNA is analyzed by PCR using primers designed to amplify a Zvn2R1 gene or a selectable marker gene that was introduced in the same plasmid. After animals are confirmed to be transgenic, they are back-crossed into an inbred strain by placing a transgenic female with a wild-type male, or a transgenic male with one or two wild-type female(s). As pups are born and weaned, the sexes are separated, and their tails snipped for genotyping.

To check for expression of a transgene in a live animal, a partial hepatectomy is performed. A surgical prep is made of the upper abdomen directly below the zyphoid process. Using sterile technique, a small 1.5-2 cm incision is made below the sternum and the left lateral lobe of the liver exteriorized. Using 4-0 silk, a tie is made around the lower lobe securing it outside the body cavity. An atraumatic clamp is used to hold the tie while a second loop of absorbable Dexon (American Cyanamid; Wayne, N.J.) is placed proximal to the first tie. A distal cut is made from the Dexon tie and approximately 100 mg of the excised liver tissue is placed in a sterile petri dish. The excised liver section is transferred to a 14 ml polypropylene round bottom tube and snap frozen in liquid nitrogen and then stored on dry ice. The surgical site is closed with suture and wound clips, and the animal's cage placed on a 37° C. heating pad for 24 hours post operatively. The animal is checked daily post operatively and the wound clips removed 7-10 days after surgery. The expression level of Zvn2R1 mRNA is examined for each transgenic mouse using an RNA solution hybridization assay or polymerase chain reaction.

In addition to producing transgenic mice that over-express Zvn2R1, it is useful to engineer transgenic mice with either abnormally low or no expression of the gene. Such transgenic mice provide useful models for diseases associated with a lack of Zvn2R1. As discussed above, Zvn2R1 gene expression can be inhibited using anti-sense genes, ribozyme genes, or external guide sequence genes. To produce transgenic mice that under-express the Zvn2R1 gene, such inhibitory sequences are targeted to Zvn2R1 mRNA. Methods for producing transgenic mice that have abnormally low expression of a particular gene are known to those in the art (see, for example, Wu et aL, “Gene Underexpression in Cultured Cells and Animals by Antisense DNA and RNA Strategies,” in Methods in Gene Biotechnology, pages 205-224 (CRC Press 1997)).

An alternative approach to producing transgenic mice that have little or no Zvn2R1 gene expression is to generate mice having at least one normal Zvn2R1 allele replaced by a nonfunctional Zvn2R1 gene. One method of designing a nonfunctional Zvn2R₁ gene is to insert another gene, such as a selectable marker gene, within a nucleic acid molecule that encodes Zvn2R1. Standard methods for producing these so-called “knockout mice” are known to those skilled in the art (see, for example, Jacob, “Expression and Knockout of Interferons in Transgenic Mice,” in Overexpression and Knockout of Vomeronasals in Transgenic Mice, Jacob (ed.), pages 111-124 (Academic Press, Ltd. 1994), and Wu et al., “New Strategies for Gene Knockout,” in Methods in Gene Biotechnology, pages 339-365 (CRC Press 1997)).

12. Therapeutic Uses of Zvn2R1 Nucleotide Sequences

The present invention includes the use of Zvn2R1 nucleotide sequences to provide Zvn2R1 to a subject in need of such treatment. In addition, a therapeutic expression vector can be provided that inhibits Zvn2R1 gene expression, such as an anti-sense molecule, a ribozyme, or an external guide sequence molecule.

There are numerous approaches to introduce a Zvn2R1 gene to a subject, including the use of recombinant host cells that express Zvn2R1, delivery of naked nucleic acid encoding Zvn2R1, use of a cationic lipid carrier with a nucleic acid molecule that encodes Zvn2R1, and the use of viruses that express Zvn2R1, such as recombinant retroviruses, recombinant adeno-associated viruses, recombinant adenoviruses, and recombinant Herpes simplex viruses (see, for example, Mulligan, Science 260:926 (1993), Rosenberg et al., Science 242:1575 (1988), LaSalle et al., Science 259:988 (1993), Wolff et al., Science 247:1465 (1990), Breakfield and Deluca, The New Biologist 3:203 (1991)). In an ex vivo approach, for example, cells are isolated from a subject, transfected with a vector that expresses a Zvn2R1 gene, and then transplanted into the subject.

In order to effect expression of a Zvn2R1 gene, an expression vector is constructed in which a nucleotide sequence encoding a Zvn2R1 gene is operably linked to a core promoter, and optionally a regulatory element, to control gene transcription. The general requirements of an expression vector are described above.

Alternatively, a Zvn2R1 gene can be delivered using recombinant viral vectors, including for example, adenoviral vectors (e.g., Kass-Eisler et al., Proc. Nat'l Acad. Sci. USA 90:11498 (1993), Kolls et al., Proc. Nat'l Acad. Sci. USA 91:215 (1994), Li et al., Hum. Gene Ther. 4:403 (1993), Vincent et al., Nat. Genet. 5:130 (1993), and Zabner et al., Cell 75:207 (1993)), adenovirus-associated viral vectors (Flotte et al., Proc. Nat'l Acad. Sci. USA 90:10613 (1993)), alphaviruses such as Semiliki Forest Virus and Sindbis Virus (Hertz and Huang, J. Vir. 66:857 (1992), Raju and Huang, J. Vir. 65:2501 (1991), and Xiong et al., Science 243:1188 (1989)), herpes viral vectors (e.g., U.S. Pat. Nos. 4,769,331, 4,859,587, 5,288,641 and 5,328,688), parvovirus vectors (Koering et al., Hum. Gene Therap. 5:457 (1994)), pox virus vectors (Ozaki et al., Biochem. Biophys. Res. Comm. 193:653 (1993), Panicali and Paoletti, Proc. Nat'l Acad. Sci. USA 79:4927 (1982)), pox viruses, such as canary pox virus or vaccinia virus (Fisher-Hoch et al., Proc. Nat'l Acad. Sci. USA 86:317 (1989), and Flexner et al., Ann. N.Y. Acad. Sci. 569:86 (1989)), and retroviruses (e.g., Baba et al., J. Neurosurg 79:729 (1993), Ram et al., Cancer Res. 53:83 (1993), Takamiya et al., J. Neurosci. Res 33:493 (1992), Vile and Hart, Cancer Res. 53:962 (1993), Vile and Hart, Cancer Res. 53:3860 (1993), and Anderson et al., U.S. Pat. No. 5,399,346). Within various embodiments, either the viral vector itself, or a viral particle which contains the viral vector may be utilized in the methods and compositions described below.

As an illustration of one system, adenovirus, a double-stranded DNA virus, is a well-characterized gene transfer vector for delivery of a heterologous nucleic acid molecule (for a review, see Becker et al., Meth. Cell Biol. 43:161 (1994); Douglas and Curiel, Science & Medicine 4:44 (1997)). The adenovirus system offers several advantages including: (i) the ability to accommodate relatively large DNA inserts, (ii) the ability to be grown to high-titer, (iii) the ability to infect a broad range of mammalian cell types, and (iv) the ability to be used with many different promoters including ubiquitous, tissue specific, and regulatable promoters. In addition, adenoviruses can be administered by intravenous injection, because the viruses are stable in the bloodstream.

Using adenovirus vectors where portions of the adenovirus genome are deleted, inserts are incorporated into the viral DNA by direct ligation or by homologous recombination with a co-transfected plasmid. In an exemplary system, the essential E1 gene is deleted from the viral vector, and the virus will not replicate unless the E1 gene is provided by the host cell. When intravenously administered to intact animals, adenovirus primarily targets the liver. Although an adenoviral delivery system with an E1 gene deletion cannot replicate in the host cells, the host's tissue will express and process an encoded heterologous protein. Host cells will also secrete the heterologous protein if the corresponding gene includes a secretory signal sequence. Secreted proteins will enter the circulation from tissue that expresses the heterologous gene (e.g., the highly vascularized liver).

Moreover, adenoviral vectors containing various deletions of viral genes can be used to reduce or eliminate immune responses to the vector. Such adenoviruses are E1-deleted, and in addition, contain deletions of E2A or E4 (Lusky et al., J. Virol. 72:2022 (1998); Raper et al., Human Gene Therapy 9:671 (1998)). The deletion of E2b has also been reported to reduce immune responses (Amalfitano et al., J. Virol. 72:926 (1998)). By deleting the entire adenovirus genome, very large inserts of heterologous DNA can be accommodated. The generation of so-called “gutless” adenoviruses, where all viral genes are deleted, is particularly advantageous for insertion of large inserts of heterologous DNA (for a review, see Yeh. and Perricaudet, FASEB J. 11:615 (1997)).

High titer stocks of recombinant viruses capable of expressing a therapeutic gene can be obtained from infected mammalian cells using standard methods. For example, recombinant herpes simplex virus can be prepared in Vero cells, as described by Brandt et al., J. Gen. Virol. 72:2043 (1991), Herold et al., J. Gen. Virol. 75:1211 (1994), Visalli and Brandt, Virology 185:419 (1991), Grau et al., Invest. Ophthalmol. Vis. Sci. 30:2474 (1989), Brandt et al., J. Virol. Meth. 36:209 (1992), and by Brown and MacLean (eds.), HSV Virus Protocols (Humana Press 1997).

Alternatively, an expression vector comprising a Zvn2R1 gene can be introduced into a subject's cells by lipofection in vivo using liposomes. Synthetic cationic lipids can be used to prepare liposomes for in vivo transfection of a gene encoding a marker (Felgner et al., Proc. Nat'l Acad. Sci. USA 84:7413 (1987); Mackey et al., Proc. Nat'l Acad. Sci. USA 85:8027 (1988)). The use of lipofection to introduce exogenous genes into specific organs in vivo has certain practical advantages. Liposomes can be used to direct transfection to particular cell types, which is particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

Electroporation is another alternative mode of administration. For example, Aihara and Miyazaki, Nature Biotechnology 16:867 (1998), have demonstrated the use of in vivo electroporation for gene transfer into muscle.

In an alternative approach to gene therapy, a therapeutic gene may encode a Zvn2R1 anti-sense RNA that inhibits the expression of Zvn2R1. Suitable sequences for anti-sense molecules can be derived from the nucleotide sequences of Zvn2R1 disclosed herein.

Alternatively, an expression vector can be constructed in which a regulatory element is operably linked to a nucleotide sequence that encodes a ribozyme. Ribozymes can be designed to express endonuclease activity that is directed to a certain target sequence in an mRNA molecule (see, for example, Draper and Macejak, U.S. Pat. No. 5,496,698, McSwiggen, U.S. Pat. No. 5,525,468, Chowrira and McSwiggen, U.S. Pat. No. 5,631,359, and Robertson and Goldberg, U.S. Pat. No. 5,225,337). In the context of the present invention, ribozymes include nucleotide sequences that bind with Zvn2R1 mRNA.

In another approach, expression vectors can be constructed in which a regulatory element directs the production of RNA transcripts capable of promoting RNase P-mediated cleavage of MRNA molecules that encode a Zvn2R1 gene. According to this approach, an external guide sequence can be constructed for directing the endogenous ribozyme, RNase P, to a particular species of intracellular MRNA, which is subsequently cleaved by the cellular ribozyme (see, for example, Altman et al., U.S. Pat. No. 5,168,053, Yuan et al., Science 263:1269 (1994), Pace et al., international publication No. WO 96/18733, George et al., international publication No. WO 96/21731, and Werner et aL, international publication No. WO 97/33991). For example, the external guide sequence can comprise a ten to fifteen nucleotide sequence complementary to Zvn2R1 mRNA, and a 3′-NCCA nucleotide sequence, wherein N is preferably a purine. The external guide sequence transcripts bind to the targeted mRNA species by the formation of base pairs between the mRNA and the complementary external guide sequences, thus promoting cleavage of mRNA by RNase P at the nucleotide located at the 5′-side of the base-paired region.

In general, the dosage of a composition comprising a therapeutic vector having a Zvn2R1 nucleotide sequence, such as a recombinant virus, will vary depending upon such factors as the subject's age, weight, height, sex, general medical condition and previous medical history. Suitable routes of administration of therapeutic vectors include intravenous injection, intraarterial injection, intraperitoneal injection, intramuscular injection, intratumoral injection, and injection into a cavity that contains a tumor. As an illustration, Horton et al., Proc. Nat'l Acad. Sci. USA 96:1553 (1999), demonstrated that intramuscular injection of plasmid DNA encoding interferon-α produces potent antitumor effects on primary and metastatic tumors in a murine model.

A composition comprising viral vectors, non-viral vectors, or a combination of viral and non-viral vectors of the present invention can be formulated according to known methods to prepare pharmaceutically useful compositions, whereby vectors or viruses are combined in a mixture with a pharmaceutically acceptable carrier. As noted above, a composition, such as phosphate-buffered saline is said to be a “pharmaceutically acceptable carrier” if its administration can be tolerated by a recipient subject. Other suitable carriers are well-known to those in the art (see, for example, Remington's Pharmaceutical Sciences, 19th Ed. (Mack Publishing Co. 1995), and Gilman's the Pharmacological Basis of Therapeutics, 7th Ed. (MacMillan Publishing Co. 1985)).

For purposes of therapy, a therapeutic gene expression vector, or a recombinant virus comprising such a vector, and a pharmaceutically acceptable carrier are administered to a subject in a therapeutically effective amount. A combination of an expression vector (or virus) and a pharmaceutically acceptable carrier is said to be administered in a “therapeutically effective amount” if the amount administered is physiologically significant. An agent is physiologically significant if its presence results in a detectable change in the physiology of a recipient subject.

When the subject treated with a therapeutic gene expression vector or a recombinant virus is a human, then the therapy is preferably somatic cell gene therapy. That is, the preferred treatment of a human with a therapeutic gene expression vector or a recombinant virus does not entail introducing into cells a nucleic acid molecule that can form part of a human germ line and be passed onto successive generations (i.e., human germ line gene therapy).

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. An isolated polypeptide, comprising an amino acid sequence selected from the group consisting of: amino acid residues 30 to 219 of SEQ ID NO:2, the amino acid sequence of SEQ ID NO:2, and the amino acid sequence of SEQ ID NO:5.

2. The isolated polypeptide of claim 1, wherein the isolated polypeptide comprises amino acid residues 30 to 219 of SEQ ID NO:2.

3. The isolated polypeptide of claim 1, wherein the isolated polypeptide comprises the amino acid sequence of SEQ ID NO:2.

4. The isolated polypeptide of claim 1, wherein the isolated polypeptide comprises the amino acid sequence of SEQ ID NO:5.

5. An isolated polypeptide, comprising the amino acid sequence of amino acid residues 30 to 610 of SEQ ID NO:8.

6. An antibody or antibody fragment that specifically binds with an amino acid sequence selected from the group consisting of:amino acid residues 30 to 610 of SEQ ID NO:8, amino acid residues 30 to 219 of SEQ ID NO:2, the amino acid sequence of SEQ ID NO:2, and the amino acid sequence of SEQ ID NO:5.

7. The antibody of claim 6, wherein the antibody is selected from the group consisting of: (a) polyclonal antibody, (b) murine monoclonal antibody, (c) humanized antibody derived from (b), and (d) human monoclonal antibody.

8. A method of identifying the presence of a Zvn2R1 ligand in a test sample, comprising: (a) contacting the test sample with a polypeptide that comprises amino acid residues 30 to 610 of SEQ ID NO:8, and (b) detecting the binding of the polypeptide to ligand in the sample.

9. The method of claim 8, wherein the polypeptide is bound to the extracellular membrane of a cultured cell.

10. The method of claim 9, wherein the ligand is contacted with a cell membrane preparation obtained from cells that produce the polypeptide.

11. An isolated nucleic acid molecule, wherein the nucleic acid molecule comprises either the nucleotide sequence of SEQ ID NO:3 or the nucleotide sequence of SEQ ID NO:6.

12. The isolated nucleic acid molecule of claim 11, comprising the nucleotide sequence of SEQ ID NO:1.

13. The isolated nucleic acid molecule of claim 11, comprising the nucleotide sequence of SEQ ID NO:4.

14. A vector, comprising the nucleotide sequence of nucleotides 88 to 1830 of SEQ ID NO:7.

15. An expression vector, comprising a nucleic acid molecule that encodes amino acid residues 30 to 610 of SEQ ID NO:8, a transcription promoter, and a transcription terminator, wherein the promoter is operably linked with the nucleic acid molecule, and wherein the nucleic acid molecule is operably linked with the transcription terminator.

16. A recombinant host cell comprising the expression vector of claim 15, wherein the host cell is selected from the group consisting of bacterium, yeast cell, avian cell, fungal cell, insect cell, mammalian cell, and plant cell.

17. A method of producing a polypeptide that comprises amino acid residues 30 to 610 of SEQ ID NO:8, the method comprising culturing recombinant host cells that comprise the expression vector of claim 15, and that produce the polypeptide.

18. The method of claim 17, further comprising isolating the polypeptide from the cultured recombinant host cells.
